(19) United States
(12) Patent Application Publication
(10) Pub. No.: US 2024/0285318 A1
(43) Pub. Date: Aug. 29, 2024

(54) ORTHOPEDIC INTRAMEDULLARY NAILS

(71) Applicants: Smith & Nephew, Inc., Memphis, TN (US); Smith & Nephew Orthopaedics AG, Zug (CH); Smith & Nephew Asia Pacific PTE. Limited, Singapore (SG)

(72) Inventors: Henry B. Faber, Memphis, TN (US); Kohsuke Watanabe, Memphis, TN (US); Adam Zysk, Arlington, TN (US); William M. Ricci, New York, NY (US); Paul Tornetta, III, Chestnut Hill, MA (US); Roy W. Sanders, Tampa, FL (US); James Krieg, Haverford, PA (US); Daniel S. Chan, Macon, GA (US); Joseph R. Hsu, Charlotte, NC (US); Benjamin Ollivere, Nottingham (GB)

(73) Assignees: Smith & Nephew, Inc., Memphis, TN (US); Smith & Nephew Orthopaedics AG, Zug (CH); Smith & Nephew Asia Pacific PTE. Limited, Singapore (SG)

(21) Appl. No.: 18/647,505

(22) Filed: Apr. 26, 2024

Related U.S. Application Data
(63) Continuation-in-part of application No. 17/921,667, filed on Oct. 27, 2022.
(63) Continuation-in-part of application No. PCT/US22/49780, filed on Nov. 14, 2022.
(60) Provisional application No. 63/020,804, filed on May 6, 2020.
(60) Provisional application No. 63/279,906, filed on Nov. 16, 2021.

(51) Int. Cl. A61B 17/72; A61B 17/56

(52) U.S. Cl. CPC A61B 17/7241; A61B 2017/564

(57) ABSTRACT

An intramedullary (“IM”) nail for internal fixation of a bone is disclosed. In one embodiment, the IM nail may be a retrograde femoral nail. Alternatively, in another embodiment, the IM nail may be a tibial IM nail. In one or more embodiments, the screw holes are arranged and configured to optimize placement of one or more screws, fasteners, or the like. In addition, and/or alternatively, an IM nail may be arranged and configured to facilitate removal of a broken screw. Alternatively, in some embodiments, the tibial IM nail may be arranged and configured to be side-specific so that the anatomic specific tibial IM nails can be used to, for example, target specific bony anatomy such as, for example, the patient's posterior malleolar, the syndesmotic joint, etc.

### CROSS-REFERENCE TO RELATED APPLICATIONS

This application is a continuation-in-part of U.S. patent application Ser. No. 17/921,667, filed on Oct. 27, 2022, which application is a National Phase filing of International Application No. PCT/US2021/030898, filed May 5, 2021, which application is a non-provisional of, and claims the benefit of the filing date of, U.S. provisional patent application No. 63/020,804, filed May 6, 2020, entitled “Orthopedic Intramedullary Nails.”

In addition, this application is a continuation-in-part of International Application No. PCT/US2022/049780, filed Nov. 14, 2022, which application is a non-provisional of, and claims the benefit of the filing date of, U.S. provisional patent application No. 63/279,906, filed Nov. 16, 2021, entitled “Anatomic Specific Orthopedic Intramedullary Tibial Nails.”

The entirety of each application is incorporated by reference herein.

### FIELD OF THE INVENTION

The present disclosure is directed to orthopedic implants (e.g., intramedullary (“IM”) nails) for stabilizing one or more patient's bones, bone portions, bone fragments, etc., and more specifically to IM nails arranged and configured to enhance screw positioning and/or removal.

In addition, in some examples, the present disclosure may be directed to anatomic specific tibial IM nails arranged and configured to be implanted within a patient's tibia to target specific bony anatomy such as, for example, the patient's posterior malleolar, while avoiding anatomic structures such as nerves, vessels, tendons, etc. In addition, the anatomic specific tibial IM nails are arranged and configured to enhance screw positioning.

### BACKGROUND

Orthopedic fixation devices (implants) may be used, for example, to stabilize an injury, to support a bone fracture, to fuse a joint, and/or to correct a deformity. Orthopedic fixation devices may be attached permanently or temporarily, and may be attached to the bone at various locations, including implanted within a canal or other cavity of the bone, implanted beneath soft tissue and attached to an exterior surface of the bone, or disposed externally and attached by fasteners such as screws, pins, and/or wires. Some orthopedic fixation devices allow the position and/or orientation of two or more bone pieces, or two or more bones, to be adjusted relative to one another. Orthopedic fixation devices are generally machined or molded from isotropic materials, such as metals including, for example, titanium, titanium alloys, stainless steel, cobalt-chromium alloys, and tantalum.

An intramedullary (“IM”) nail is one type of orthopedic fixation device. The primary function of the IM nail is to stabilize the fracture fragments, and thereby enable load transfer across the fracture site while maintaining anatomical alignment of the bone. Currently, there are a large number of different commercially available IM nails in the marketplace.

One known type of an IM nail is a retrograde femoral nail. A retrograde femoral nail is arranged and configured to be inserted into the medullary canal of a patient's femur through the distal end of the femur (e.g., patient's knee). In use, the retrograde femoral nail may extend from the patient's knee proximally to the proximal end portion of the patient's femur (e.g., adjacent or near the greater trochanter).

One disadvantage with known retrograde femoral nails is that placement or targeting of a screw or fastener (terms used interchangeably herein without the intent to limit) in the medial-lateral direction at the proximal end portion of the retrograde femoral nail is rendered difficult or impossible (e.g., standard fluoroscopic machines used to target fastener placement will not fit around an upper thigh of a patient). As a result, retrograde femoral nails lack a screw opening, hole, slot, or the like in the medial-lateral direction at the proximal end portion thereof.

Another disadvantage with known retrograde femoral nails is that screw holes formed in the distal end portion of the retrograde femoral nail may not be properly aligned in situ to secure certain bone fragments relative to the retrograde femoral nail. In addition, and/or alternatively, known retrograde femoral nails lack the ability to perform compression. For example, known retrograde femoral nails incorporate first, second, and third screw holes or openings in the distal end portion thereof, and do not include an elongated slot arranged and configured to enable locking compression.

Another known type of an IM nail is a tibial IM nail. A tibial IM nail is arranged and configured to be inserted into the medullary canal of a patient's tibia. In use, the proximal end portion of a tibial IM nail needs to allow for adequate fragment fixation near the tibia plateau without penetrating through the tibia plateau. In addition, in use, fixation of the distal end portion of the tibial IM nail should avoid anatomic structures such as nerves, vessels, tendons, etc. To accomplish this, known tibial IM nails incorporate a single distal hole formed in the distal end portion, the hole extending in the anterior-posterior direction. In addition, the distal end portion of the tibial IM nail may include two oblique holes. In use, the oblique holes may extend in different radial directions and enclose different angles with one another.

One concern with existing tibial IM nails is that their distal end portions are not arranged and configured to provide increased fixation while avoiding anatomic structures. That is, it would be beneficial for the distal end portion of a tibial IM nail to include screw holes arranged and configured to project the screws along trajectories that are less likely to interact with anatomic structures (e.g., nerves, vessels, and tendons).

In addition, the proximal end portion of the tibia IM nail include holes that are arranged and configured so that the proximal most screws in the proximal end portion are typically positioned in holes that are perpendicular to a central longitudinal axis of the proximal portion of the tibial IM nail. However, in use, because the central longitudinal axis of the proximal portion of the tibia IM nail is not necessarily positioned in a tibia perpendicularly with the tibial plateau, the proximal most screws may not be parallel to the tibial plateau and may even penetrate the tibial plateau. Non-parallel placement of screws with the tibial plateau is not idea and penetration of the tibial plateau is unacceptable.

One known solution to ensure that the most proximal screw is implanted substantially parallel with the tibial plateau is to incorporate a supplemental component to the proximal end portion of the tibial IM nail to achieve placement of the most proximal fastener substantially parallel with the tibial plateau. However, this requires additional instrumentation, method steps, and time to position the nail and then attach the supplemental component to the nail.

Another concern or disadvantage of current tibial IM nails is that they are designed and configured to be side agnostic. That is, current tibial IM nails are identical to each other (e.g., current tibial IM nails are identical regardless if being implanted within the patient's right tibia or left tibia). That is, to date, there are no mirror-image tibial nails, where the two nails cannot be overlaid on each other with a perfect fit. As a result, targeting specific bony landmarks in or adjacent to the patient's tibia is nearly impossible. For example, if a current tibial IM nail was used to target a specific landmark in the patient's left tibial, the same tibial IM nail implanted into the patient's right tibia would be incapable of targeting the landmark.

Thus, for example, current tibial IM nails are incapable of targeting the patient's posterior malleolar. Generally speaking, posterior malleolar fractures are poorly treated as they may go undetected on X rays because they are spiral type fractures. In addition, even when detected, posterior malleolar fractures are difficult to treat. Current tibial IM nails are incapable of targeting posterior malleolar fractures. As a result, posterior malleolar fractures are typically treated using bone plates and/or screws, which are designated as a separate procedure. It would be beneficial for the distal end portion of the tibial IM nail to enable fixation of posterior malleolar fractures.

It would also be beneficial for the distal end portion of the tibial IM nail to enable fixation of across the syndesmotic joint. Once again, current tibial IM nails do not adequately accommodate fixation across the syndesmotic joint into the patient's fibula. Once again, by being side agnostic, current tibial IM nails have inadequate screw trajectories to accommodate side specific points of fixation. As a result, current tibial IM nails do not enable fixation across the syndesmosis in both limbs (e.g., if current tibial IM nails included a screw trajectory that enable fixation across the syndesmosis in one limb such as, for example, the patient's left tibia, the tibial IM nail wouldn't enable fixation across the syndesmosis in the contralateral limb such as, for example, the patient's right tibia—current tibial IM nails do not enable targeting in both limbs).

Finally, it would be beneficial for any tibial IM nail to be arranged and configured so that the driving end of screws used for fixation can be inserted such that they are not too proud of the bone, as this can cause irritation to the patient.

Thus, there remains a need to provide improved orthopedic IM nails for internal fixation of a bone. The present invention satisfies this need and provides other benefits and advantages in a novel and unobvious manner.

### BRIEF SUMMARY

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

In one embodiment, a retrograde intramedullary femoral nail is disclosed. In use, the retrograde intramedullary femoral nail is arranged and configured to be implanted into a patient's femur via a distal end of the femur. The retrograde intramedullary femoral nail includes a body including a leading proximal end portion and a distal end portion opposite the leading proximal end portion. The leading proximal end portion includes a plurality of anterior-posterior screw holes or openings arranged and configured to receive a screw or fastener in the anterior-posterior direction in situ. In addition, the leading proximal end portion includes a medial-lateral screw hole or opening arranged and configured to receive a screw or fastener in the medial-lateral direction in situ so that the medial-lateral screw hole or opening is arranged perpendicular to the plurality of anterior-posterior screw holes or openings.

In one embodiment, the medial-lateral screw hole or opening is in the form of an elongated slot.

In one embodiment, the leading proximal end portion includes first and second anterior-posterior screw holes or openings extending in the anterior-posterior direction for receiving first and second screws or fasteners, respectively. The medial-lateral elongated screw slot being positioned in-between the first and second anterior-posterior screw holes or openings extending in the anterior-posterior direction.

In one embodiment, the retrograde intramedullary femoral nail is provided in a system or kit with a targeting system such as, for example, an electromagnetic field tracking system. For example, the retrograde intramedullary femoral nail may be provided with the SureShot targeting system manufactured and sold by Smith Nephew, Inc. In use, the SureShot targeting system is a computer-based software system that provides perfect circle targeting to provide continuous visual real-time feedback of drill position to ensure correct direction and angle. Utilizing the SureShot targeting system, the need for fluoroscopy during targeting of the medial-lateral screw opening, hole or slot is eliminated.

In one embodiment, the retrograde intramedullary femoral nail is implanted into the medullary canal of a patient's femur via the distal end or knee. Once properly implanted, one or more screws or fasteners can be inserted into the proximal end portion of the retrograde intramedullary femoral nail utilizing the SureShot targeting system. In one embodiment, a screw or fastener can be positioned and inserted into the medial-lateral slot formed in the proximal end portion of the retrograde intramedullary femoral nail. In addition, and/or alternatively, one or more screws or fasteners may be inserted into the anterior-posterior openings, holes, or slots formed in the proximal end portion of the retrograde intramedullary femoral nail.

In one embodiment, a retrograde femoral nail is disclosed. The retrograde femoral nail arranged and configured to be implanted into a patient's femur via a distal end of the femur. The retrograde intramedullary femoral nail includes a body including a leading proximal end portion and a distal end portion opposite the leading proximal end portion. The distal end portion includes a plurality of screw holes or openings arranged and configured to receive a screw or fastener. In one embodiment, the distal end portion of the retrograde femoral nail includes first, second, third, and fourth holes or openings, each of the first, second, third, and fourth screw holes or openings arranged and configured to receive a screw or fastener.

In one embodiment, the first screw hole or opening (e.g., screw hole or opening positioned closest to the distal end of the retrograde femoral nail) extends in a substantially medial-lateral direction in situ. Meanwhile, in one embodiment, the second, third and fourth holes or openings are angled or oblique relative to the first screw hole or opening. In one embodiment, the second, third, and fourth holes or openings are angled between about ±15 degrees to about ±45 degrees relative to a medial-lateral plane. Alternatively, in one embodiment, the fourth hole or opening may be substantially parallel to the first screw hole or opening, and thus extend in a substantially medial-lateral direction in situ. The second and third holes or openings being angled or oblique and may be angled between about ±15 degrees to about ±45 degrees relative to a medial-lateral plane.

In one embodiment, one or both of the second and third screw holes or openings are elongated slots. In use, a reduction instrument may be used in connection with the one or more elongated slots to compress the fracture in the patient's bone.

In one embodiment, a tibial IM nail is disclosed. The tibial IM nail is arranged and configured to be implanted into the medullary canal of a patient's tibia. The tibial IM nail includes a body including a leading distal end portion and a proximal end portion opposite the leading distal end portion. In one embodiment, the distal end portion includes a plurality of screw openings, holes, slots, etc. arranged and configured to receive a screw or fastener. In one embodiment, the distal end portion of the tibial IM nail includes first, second, third, and fourth screw holes or openings. The second and third screw holes or openings may be arranged and configured to extend at oblique angles relative to the medial-lateral plane.

In one embodiment, the first and fourth screw holes or openings extend substantially in the medial-lateral direction. As such, the second and third screw holes or openings are arranged and configured at an oblique angle relative to the first and fourth screw holes or openings.

In one embodiment, the second and third screw holes or openings are arranged and configured at an oblique angle α between approximately ±30 to ±60 degrees.

In one embodiment, the second and third oblique screw holes or openings are also tilted (e.g., angled vertically). That is, the second and third screw holes or openings are vertically angulated relative to a central longitudinal axis of the body so that a screw or fastener passing through the second and third screw holes or openings extend through the body at a vertical angle.

In one embodiment, a tibial IM nail is disclosed. The tibial IM nail includes a body including a leading distal end portion and a proximal end portion opposite the leading distal end portion. The proximal end portion includes a plurality of screw openings, holes, or slots, each of the plurality of screw holes or openings arranged and configured to receive a screw or fastener. In one embodiment, the first most proximal screw hole or opening is arranged and configured to align a screw or fastener so that a screw or fastener passing through the first or most proximal screw hole or opening does not penetrate the patient's tibial plateau TP through which the tibial IM nail is inserted. That is, in one embodiment, the first or most proximal screw hole or opening formed in the proximal end portion of the tibia IM nail is tilted or angled downwards away from the proximal end of the tibial IM nail so that the screw or fastener inserted therein does not penetrate the tibial plateau TP (e.g., the first most proximal screw hole or opening includes a longitudinal central axis that is downwardly horizontally angled relative to a longitudinal central axis of the body so that the first most proximal screw hole or opening is angled downwards away from the proximal end portion). In one embodiment, the screw or fastener may be positioned substantially parallel to the tibial plateau TP.

In one embodiment, the first or proximal most screw hole or opening is angled or tilted downwards by approximately 5 to 10 degrees.

In one embodiment, the first or proximal most screw hole or opening is angled or tilted downwards by approximately 5 degrees.

In one embodiment, the first or proximal most screw hole or opening is angled or tilted downwards by approximately 7 to 10 degrees.

In one embodiment, the proximal end portion of the tibial IM nail includes additional screw holes or openings including, for example, second, third and fourth screw holes or openings. In one embodiment, the second screw hole or opening from the proximal end of the tibia IM nail is also tilted or angled. In one embodiment, the second screw hole or opening includes a downward tilt or angle. In another embodiment, the second screw hole or opening includes an upward tilt or angle.

In one embodiment, the third screw hole or opening from the proximal end of the tibial IM nail is in the form of an elongated slot.

In one embodiment, an IM nail is disclosed. The IM nail includes a body including a distal end portion, a proximal end portion opposite the distal end portion, and a screw hole or opening extending through a first wall of the IM nail and a second wall of the IM nail. The screw hole portion extending through the first wall is threaded, the screw hole portion extending through the second wall is devoid of any threads (e.g., non-threaded). That is, the screw hole extends through a first wall of the body and a second wall of the body, the screw hole defining a proximal screw hole portion passing through the first wall and a distal screw hole portion passing through the second wall, the proximal screw hole portion comprising threads and the distal screw hole portion being non-threaded.

In one embodiment, the screw hole portion formed in the second wall includes a diameter D2 that is greater than a diameter D1 of the threaded portion of the locking screw so that the threaded portion of the screw can pass through the screw hole portion formed in the second wall. That is, the proximal screw hole portion comprises a diameter D1 and the distal screw hole portion comprises a diameter D2 that is greater than the diameter D1. Alternatively, in one embodiment, diameter D2 may be equal to diameter D1.

Alternatively, an anatomic, side-specific tibial IM nail is also disclosed. In some examples, the tibial IM nail includes a body, a proximal end portion, and a distal end portion. The distal end portion includes a plurality of screw holes arranged and configured to receive a fastener. The plurality of screw holes including a distal most, first screw hole. The distal most, first screw hole being angled or inclined in a superior-inferior direction relative to a central longitudinal axis of the distal end portion. In some examples, the distal most, first screw hole may be angled or inclined by an angle α with angle α being approximately ten-degrees so that a first screw positioned within the distal most, first screw hole can target a patient's posterior malleolus or other bony anatomy.

In some of the preceding or subsequent examples, the plurality of screw holes formed in the distal end portion further include second, third, and fourth screw holes positioned from a distal end of the body.

In some of the preceding or subsequent examples, the second screw hole and the fourth screw hole extend substantially parallel to each other. In some examples, the second screw hole and the fourth screw hole extend substantially parallel to an anterior-posterior plane in situ.

In some of the preceding or subsequent examples, the distal most, first screw hole may be angled by an angle ∞ in a medial-lateral direction. In some examples, the angle ∞ is about ±70 degrees from the anterior-posterior plane (or relative to second and fourth screw holes). In some examples, the angle ∞ is about ±62.5 degrees from the anterior-posterior plane (or relative to second and fourth screw holes).

In some of the preceding or subsequent examples, the third screw hole may be angled by an angle Ω in the medial-lateral direction. In any preceding or subsequent example, the angle Ω is about ±30 degrees from the anterior-posterior plane (or relative to the second and fourth screw holes).

Alternatively, an anatomic, side-specific tibial IM nail is also disclosed. In some examples, the tibial IM nail includes a body, a proximal end portion, and a distal end portion. The proximal end portion includes a plurality of screw holes arranged and configured to receive a fastener. The plurality of screw holes including a proximal most, first screw hole. The proximal most, first screw hole includes a downward angle or inclination of angle μ in a superior-inferior direction. In some examples, angle μ is approximately 10 degrees.

In some of the preceding or subsequent examples, the proximal end portion further includes a second screw hole positioned from a proximal end of the body. In some examples, the first and second screw holes are arranged and configured to project, cross, exit, or the like from an outer surface of the patient's tibia at substantially the same level or plane. In some examples, the second screw hole is substantially perpendicular to a central longitudinal axis of the proximal end portion.

In some of the preceding or subsequent examples, the proximal end portion further includes third and fourth screw holes positioned from the proximal end of the body.

In some of the preceding or subsequent examples, one or more of the plurality of screw holes is arranged and configured as a slotted hole.

In some of the preceding or subsequent examples, the third screw opening is arranged and configured as a slotted hole.

In some of the preceding or subsequent examples, the third screw hole extends substantially in a medial-lateral direction in situ.

In some of the preceding or subsequent examples, the proximal most, first screw hole and the second screw hole are angled in the medial-lateral plane by an angle § 1, § 2, respectively, and thus relative to the third screw hole. In some examples, angles § 1, § 2 are identical. In some examples, angles § 1, § 2 are about ±47.5 degrees. In some examples, angle § 1 is different from angle § 2. In some examples, angle § 1 is about ±44 degrees while angle § 2 is about ±47.5 degrees.

In some of the preceding or subsequent examples, the fourth screw hole is angled or inclined in the superior-inferior direction by an angle £. In some examples, angle £ is about ±5 degrees.

In some of the preceding or subsequent examples, the fourth screw hole is angled in the medial-lateral plane and thus relative to the third screw hole by an angle Σ. In some examples, angle Σ is about ±25 degrees. Alternatively, in some examples, angle Σ is about ±35 degrees. Alternatively, in some examples, angle Σ is about ±40 degrees.

In some of the preceding or subsequent examples, the proximal end region includes a fifth screw hole.

In some of the preceding or subsequent examples, the fifth screw hole extends in the medial-lateral plane, and thus parallel to the third screw hole. In some examples, the third screw hole and the fifth screw hole are separated from each other by the fourth screw hole positioned between the third and fifth screw holes.

In some examples, an anatomic, side-specific tibial IM nail is also disclosed. The anatomic, side-specific tibial IM nail being arranged and configured to be implanted within one of a patient's right tibial and a patient's left tibial, but not the other one of the patient's right and left tibial.

In some examples, a kit or set of tibial IM nails is also disclosed. In some examples, the kit or set includes a plurality of tibial IM nails including a right tibial IM nail and a left tibial IM nail, wherein the right and left tibial IM nails are mirror-images of each other. That is, the kit or set of tibial IM nails includes mutual sets of side-specific anatomic tibial IM nails wherein one tibial IM nail is arranged and configured for implantation into the patient's left tibia and one of the tibial IM nails is arranged and configured for implantation into the patient's right tibia.

In some examples, a method for treating a patient's posterior malleolar fracture is also disclosed. In some examples, the method includes selecting a side-specific tibial IM nail from a plurality of tibial IM nails including a right tibial IM nail and a left tibial IM nail, inserting the selected tibial IM nail into the patient's intramedullary canal, and targeting the patient's posterior malleolar fracture via a fastener inserted through a screw hole formed in a distal end portion of the selected tibial IM nail. For example, in some examples, the method for treating a patient's posterior malleolar fracture includes selecting a corresponding side-specific tibial IM nail from a plurality of tibial IM nails including a right tibial IM nail and a left tibial IM nail depending on a location of the fracture, implanting the selected side-specific tibial IM nail into the patient's intramedullary canal, and inserting a fastener through a screw hole formed in a distal end portion of the side-specific tibial IM nail, the fastener targeting the patient's posterior malleolar fracture.

In some examples, a method for treating a patient's syndesmotic joint is also disclosed. In some examples, the method includes selecting a side-specific tibial IM nail from a plurality of tibial IM nails including a right tibial IM nail and a left tibial IM nail, inserting the selected tibial IM nail into the patient's intramedullary canal, and targeting the patient's syndesmotic joint via a fastener inserted through a screw hole formed in a distal end portion of the selected tibial IM nail. For example, the method for treating a patient's syndesmotic joint fracture includes selecting a corresponding side-specific tibial IM nail from a plurality of tibial IM nails including a right tibial IM nail and a left tibial IM nail depending on a location of the fracture, implanting the selected side-specific tibial IM nail into the patient's intramedullary canal, and inserting a fastener through a screw hole formed in a distal end portion of the side-specific tibial IM nail, the fastener targeting the patient's syndesmotic joint fracture.

Embodiments of the present disclosure provide numerous advantages. For example, by incorporating one or more features of the present disclosure, the proximal end of a retrograde femoral nail may be fixated in the medial-lateral direction (in situ) to provide better securement, the distal end of a retrograde femoral nail may be provided with additional, oblique screw holes for enabling coupling to potentially more fracture fragments, the distal end of a tibial IM nail may include additional screw holes arranged and configured to avoid anatomic structures, and the proximal end of a tibial IM nail may be arranged and configured to avoid penetration of the tibial plateau. In addition, in accordance with another feature of the present disclosure, the screw hole may be arranged and configured to facilitate easier removal of a distal portion of a broken locking screw. For example, the screw hole may be arranged and configured to enable the distal portion of a broken screw to be removed through the screw hole.

Alternatively, in some examples, by designing and providing tibial IM nails in anatomically specific configurations, the tibial IM nails can be arranged and configured to target specific bony anatomy such as, for example, the patient's posterior malleolar fracture, syndesmotic joint, etc., which heretofore have not been possible with tibial IM nails.

Further features and advantages of at least some of the embodiments of the present invention, as well as the structure and operation of various embodiments of the present invention, are described in detail below with reference to the accompanying drawings.

### BRIEF DESCRIPTION OF THE DRAWINGS

By way of example, a specific embodiment of the disclosed device will now be described, with reference to the accompanying drawings, in which:

FIG. 1 is a perspective view of an example of an embodiment of a retrograde femoral intramedullary (“IM”) nail in accordance with one or more features of the present disclosure;

FIG. 2 is an alternate perspective view of the retrograde femoral IM nail shown in FIG. 1;

FIG. 3 is a side view of the retrograde femoral IM nail shown in FIG. 1;

FIG. 4 is an alternate side view of the retrograde femoral IM nail shown in FIG. 1;

FIG. 5 is a detailed view of a proximal end portion of the retrograde femoral IM nail shown in FIG. 1;

FIG. 6 is a detailed view of a distal end portion of the retrograde femoral IM nail shown in FIG. 1;

FIG. 7 is a perspective view of an example of an embodiment of a tibial IM nail in accordance with one or more features of the present disclosure;

FIG. 8 is a side view of the tibial IM nail shown in FIG. 7;

FIG. 9 is an alternate side view of the tibial IM nail shown in FIG. 7;

FIG. 10A is a detailed view of a distal end portion of the tibial IM nail shown in FIG. 7;

FIG. 10B is an alternate detailed view of a distal end portion of the tibial IM nail shown in FIG. 7;

FIG. 11A is a detailed view of a proximal end portion of the tibial IM nail shown in FIG. 7;

FIG. 11B is an alternate detailed view of the proximal end portion of the tibial IM nail shown in FIG. 7;

FIG. 12 is a cross-sectional view of a screw hole formed in an IM nail in accordance with one or more features of the present disclosure;

FIG. 13A is a perspective view of an example of a tibial intramedullary (“IM”) nail in accordance with one or more features of the present disclosure, the tibial IM nail shown implanted within a patient's left tibia;

FIG. 13B is an alternate perspective view of the tibial IM nail shown in FIG. 13A;

FIG. 14 is a detailed, perspective view of a distal end portion or region of the tibial IM nail shown in FIG. 13A;

FIG. 15 is a top-down view of the distal end portion or region of the tibial IM nail shown in FIG. 13A;

FIG. 16 is a detailed, perspective view of a proximal end portion or region of the tibial IM nail shown in FIG. 13A;

FIG. 17 is a top-down view of the proximal end portion or region of the tibial IM nail shown in FIG. 13A;

FIGS. 18A-18D illustrate various views of an alternate example of a proximal end portion or region of a tibial IM nail in accordance with one or more features of the present disclosure;

FIGS. 19A-19D illustrate various views of an alternate example of a proximal end portion or region of a tibial IM nail in accordance with one or more features of the present disclosure;

FIGS. 20A-20D illustrate various views of an alternate example of a proximal end portion or region of a tibial IM nail in accordance with one or more features of the present disclosure;

FIG. 21 illustrates an alternate example of a tibial IM nail in accordance with one or more features of the present disclosure;

FIGS. 22A and 22B illustrate various views of a screw inserted through a first screw hole formed in the distal end portion or region of the tibial IM nail shown in FIG. 13A, the screw being used to target a patient's posterior malleolus in accordance with one or more features of the present disclosure; and

FIGS. 23A and 23B illustrate various views of a screw inserted through an alternate or third screw hole formed in the distal end portion or region of the tibial IM nail shown in FIG. 13A, the screw being used to target a patient's syndesmotic joint in accordance with one or more features of the present disclosure.

It should be understood that the drawings are not necessarily to scale and that the disclosed embodiments are sometimes illustrated diagrammatically and in partial views. In certain instances, details which are not necessary for an understanding of the disclosed methods and devices or which render other details difficult to perceive may have been omitted. It should be further understood that this disclosure is not limited to the particular embodiments illustrated herein. In the drawings, like numbers refer to like elements throughout unless otherwise noted.

### DETAILED DESCRIPTION

Various features or the like of IM nails will now be described more fully hereinafter with reference to the accompanying drawings, in which one or more features of the IM nails will be shown and described. It should be appreciated that the various features or the like may be used independently of, or in combination, with each other. It will be appreciated that an IM nail as disclosed herein may be embodied in many different forms and should not be construed as being limited to the embodiments set forth herein. Rather, these embodiments are provided so that this disclosure will convey certain features of the IM nails to those skilled in the art.

Disclosed herein are various IM nails including one or more features arranged and configured to optimize placement of one or more screws, fasteners, or the like (terms used interchangeably herein without the intent to limit). In addition, and/or alternatively, disclosed herein is an IM nail including one or more features arranged and configured to facilitate removal of a broken screw.

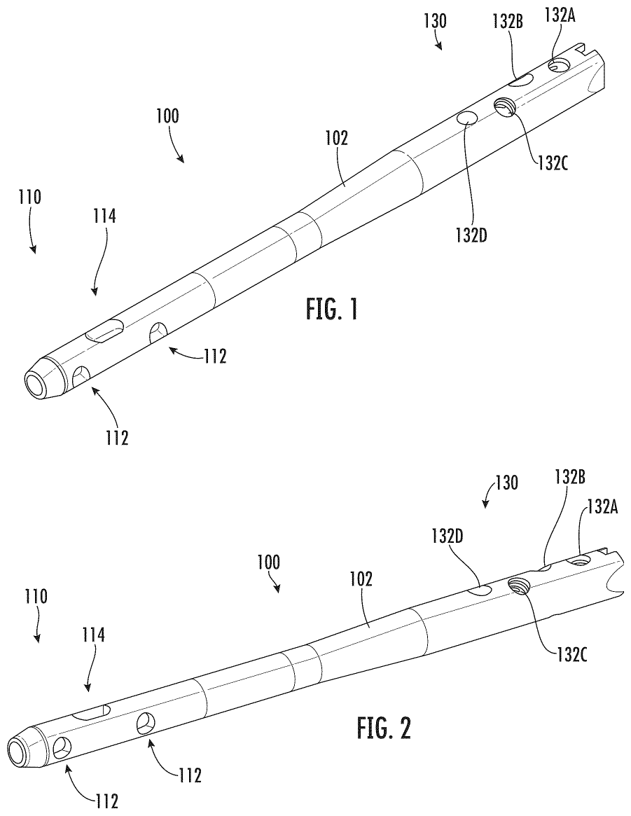

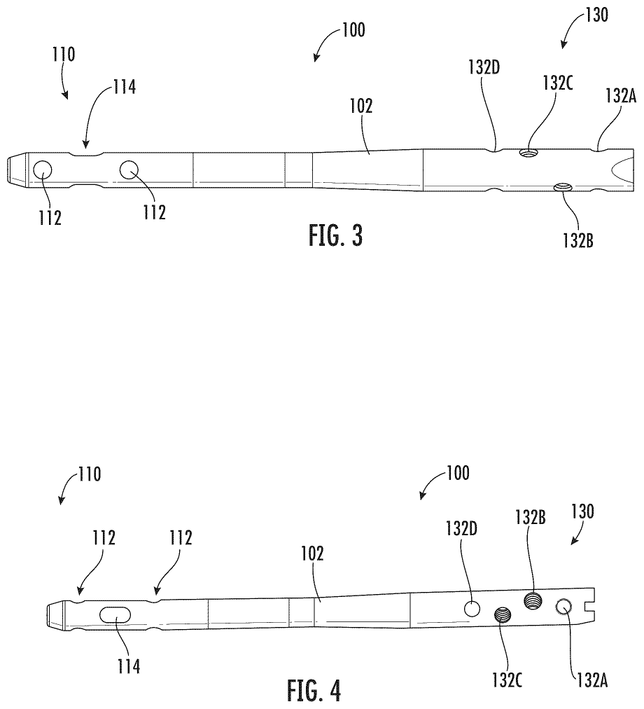

Referring to FIGS. 1-4, an example of an embodiment of a retrograde IM nail 100 in accordance with one or more features of the present disclosure is illustrated. In use, as previously mentioned, a retrograde IM nail 100 is arranged and configured to be implanted into the medullary canal of a patient's femur via the distal end of the patient's femur (e.g., via the patient's knee). As such, the retrograde IM nail 100 may also be referred to as a retrograde femoral nail.

As illustrated, the retrograde femoral nail 100 includes a body 102 such as, for example, a cannulated body. The body 102 includes a leading or proximal end portion 110 (leading proximal end portion) and a distal end portion 130. With reference to FIGS. 1-5, in accordance with one or more features of the present disclosure, the proximal end portion 110 includes a plurality of screw openings, holes, slots, etc. 112 arranged and configured to receive a fastener, screw, etc. (terms used interchangeably herein without the intent to limit) in the anterior-posterior direction in situ. In one embodiment, the screw openings, holes, slots, etc. can be threaded. Alternatively, the screw openings, holes, slots, etc. can be non-threaded, or have any other configuration now known or hereafter developed. As illustrated, in one embodiment, the proximal end portion 110 includes first and second screw holes 112, although more or less screw holes may be incorporated.

In addition, as illustrated, the proximal end portion 110 of the retrograde femoral nail 100 includes a screw opening, hole, slot, etc. 114 arranged and configured to receive a screw in the medial-lateral direction in situ. Thus arranged, the medial-lateral screw hole or slot 114 may extend in a direction substantially perpendicular to the first and second screw holes 112. In one embodiment, the medial-lateral screw hole 114 is in the form of a slot. By utilizing a slot, dynamization or micro-motion of the retrograde femoral nail 100 in situ is enabled.

Alternatively, and/or in addition, in one embodiment, the medial-lateral slot 114 may be utilized to enable a surgeon to position a fastener into a femoral neck and head of the patient's femur. In one embodiment, the retrograde femoral nail 100 would have a length sufficient to extend the retrograde femoral nail 100 into the patient's femur to target the femoral neck and head area. The medial-lateral slot 114 may have a larger size or height to enable improved positioning and/or angulation of the fastener into the femoral neck and head. In addition, in one embodiment, the medial-lateral screw hole or slot 114 may be non-perpendicular to the first and second screw holes 112. Thus arranged, the medial-lateral screw hole or slot 114 may be arranged and configured to enable some anteversion. In connection with this embodiment, the medial-lateral slot 114 may be positioned as proximal as possible to the proximal leading edge (e.g., the medial-lateral slot 114 may take the position of the first screw hole 112). Thus arranged, by positioning the medial-lateral slot 114 adjacent to the proximal leading edge, the medial-lateral slot 114 may provide enhanced neck targeting. In addition, and/or alternatively, it is envisioned that the retrograde femoral nail 100 may also include a second slot (e.g., one or more of the screw holes 112 may be converted to a slot). In addition, and/or alternatively, the slots need not extend purely in the medial-lateral direction.

As illustrated, the proximal end portion 110 of the retrograde femoral nail 100 may include first and second screw holes 112 extending in the anterior-posterior direction for receiving first and second screws, respectively. In one embodiment, the medial-lateral screw hole or slot 114 may be positioned in-between the first and second screw holes 112 extending in the anterior-posterior direction. Although this is but one example and it should be understood that the proximal end portion 110 of the retrograde femoral nail 100 may include more or less screw holes extending in the anterior-posterior direction and more screw holes or slots extending in the medial-lateral direction. In addition, the screw holes may include alternate configurations such as, for example, the screw hole or slot extending in the medial-lateral direction may be positioned above or below (e.g., proximal or distal) of the screw holes extending in the anterior-posterior direction.

As illustrated, in one embodiment, the first or proximal most anterior-posterior screw hole 112 may be positioned as close as possible to the leading proximal end of the retrograde femoral nail 100. For example, the first or proximal most anterior-posterior screw hole 112 may be positioned approximately 5 mm to 10 mm from the proximal end of the retrograde femoral nail 100. The medial-lateral screw hole or slot 114 may be positioned such that a center of a proximal end of the medial-lateral screw hole or slot 114 is at least 7 mm from the first or proximal most anterior-posterior screw hole 112. In addition, the second anterior-posterior screw hole 112 may be positioned at least 7 mm from a center of a distal end of the medial-lateral screw hole or slot 114.

Alternatively, and/or additionally, in one embodiment, the medial-lateral screw hole or slot 114 may be positioned approximately 25 mm from the proximal end of the retrograde nail 100 and the third screw hole may be positioned approximately 35 mm from the proximal end of the retrograde femoral nail 100, although these dimensions are merely example and other dimensions can be used. Preferably, in one embodiment, the first, second, and third screw holes 112, 114 are positioned as close as possible while maintaining structural integrity of the retrograde femoral nail 100. In one embodiment, the first, second, and third screw holes 112, 114 are positioned within 40 mm extending from the leading proximal end of the retrograde femoral nail 100.

By providing a medial-lateral screw opening, hole, or slot 114 in the proximal end portion 110 of a retrograde femoral nail 100 improved fixation of the proximal end portion 110 of the retrograde femoral nail 100 can be achieved. In addition, and/or alternatively, providing one or more medial-lateral screw openings, holes, or slots 114 in the proximal end portion 110 of a retrograde femoral nail 100 facilitates easier connection to a bone plate via, for example, a screw passing through the bone plate and through the medial-lateral screw opening, hole, or slot.

In use, the medial-lateral screw opening, hole, or slot 114 can be targeted (e.g., located) utilizing, for example, a targeting device that includes a low-profile medial component. For example, the medial-lateral screw opening, hole or slot 114 can be targeted utilizing electromagnetic field tracking capabilities of the SureShot targeting system manufactured and sold by Smith Nephew, Inc. Alternatively, in an alternate embodiment, it is envisioned that the medial-lateral screw opening, hole or slot 114 formed in the proximal end portion 110 of the retrograde femoral nail 100 could be targeted utilizing an instrument that indexes from one or both of the screw holes 112 extending in the anterior-posterior direction.

In use, SureShot targeting system is a computer-based software system that provides perfect circle targeting providing continuous visual real-time feedback of drill position to ensure correct direction and angle. Utilizing SureShot targeting system, need for fluoroscopy during targeting of the medial-lateral screw opening, hole, or slot 114 in the retrograde femoral nail 100 is eliminated. Given the unique slimline operation of the SureShot targeting system, the limitations of placing fluoroscopic equipment on the medial side of the retrograde femoral nail 100 (e.g., in patient's crotch area) can be overcome.

In one embodiment, in use, the SureShot targeting system includes a field generator for generating one or more magnetic fields, a removable probe with a first magnetic sensor, a landmark identifier, and a processor. The landmark identifier may include a second sensor, or, alternatively, the field generator. The processor can utilize sensor data and, if desirable, field generator and other information, to generate and display a position and orientation of the sensor(s) in preferably six degrees of freedom, and thereby, to generate and display the position and orientation of a landmark (e.g., screw hole) formed in an orthopaedic implant (e.g., retrograde femoral nail) positioned within the magnetic fields. The system allows for blind targeting of one or more landmarks. Additional information on the SureShot targeting system can be found in U.S. Pat. No. 8,623,023, filed Feb. 18, 2011, entitled Targeting an Orthopaedic Implant Landmark, the entire disclosure of which is hereby incorporated by reference in its entirety.

In one embodiment, the retrograde femoral nail 100 can be provided in a system. The system may include one or more retrograde femoral nails 100 including a medial-lateral screw opening, hole, or slot 114 formed in the proximal end portion 110 thereof. The system may further include a SureShot targeting system for identifying and placing a screw through the medial-lateral opening, hole, or slot 114. For example, in one embodiment, a retrograde femoral nail 100 may be implanted into the medullary canal of a patient's femur via the distal end or knee. Once properly implanted, one or more screws can be inserted into the proximal end portion 110 of the retrograde femoral nail 100 utilizing the SureShot targeting system. For example, a screw can be positioned and inserted into the medial-lateral slot 114 formed in the proximal end portion 110 of the retrograde femoral nail 100. In addition, and/or alternatively, one or more screws may be inserted into the anterior-posterior openings, holes, or slots 112 formed in the proximal end portion 110 of the retrograde femoral nail 100.

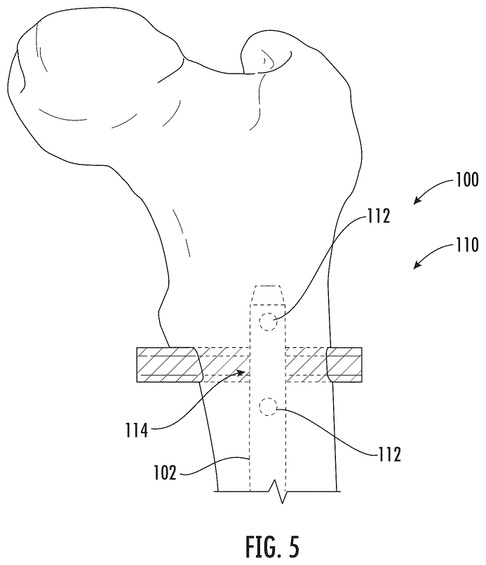

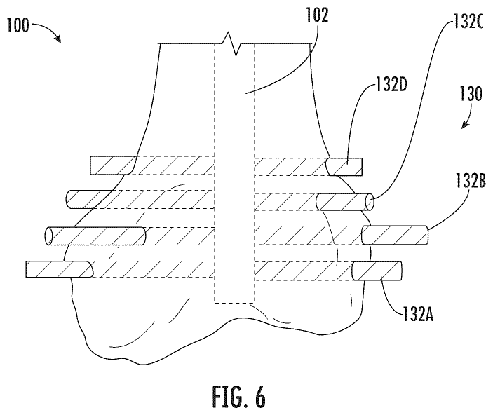

Referring to FIGS. 1-4 and 6, in accordance with one or more features of the present disclosure that may be used separately from or in combination with the novel proximal end portion 110 of the retrograde femoral nail 100 discussed above in connection with FIG. 5, the distal end portion 130 of the retrograde femoral nail 100 includes a plurality of screw openings, holes, slots, etc. 132 arranged and configured to receive a screw. As illustrated, the distal end portion 130 of the retrograde femoral nail 100 includes first, second, third, and fourth holes 132A, 132B, 132C, 132D.

In one embodiment, the first hole 132A may be positioned approximately 10 mm from the distal end of the retrograde femoral nail 100, the second hole 132B may be positioned approximately 20 mm from the distal end, the third hole 132C may be positioned approximately 30 mm from the distal end, and the fourth hole 132D may be positioned approximately 40 mm from the distal end, although these dimensions are merely examples and other dimensions can be used.

Thus arranged, by incorporating an additional or fourth screw hole in the distal end portion 130 of the retrograde femoral nail 100, additional screw fixation may be achieved enabling fixation to potentially more bone fragments.

In one embodiment, the first screw hole 132A (e.g., screw hole closest to the distal end of the retrograde femoral nail 100) may extend in substantially the medial-lateral direction in situ. Meanwhile, in one embodiment, the second, third and fourth screw holes 132B, 132C, 132D may be angled or oblique. For example, the second, third, and fourth screw holes 132B, 132C, 132D may be angled between about ±15 degrees to about ±45 degrees, preferably about ±25 degrees, from the medial-lateral plane (or relative to the first screw hole 132A), although other angles may be used. Alternatively, in one embodiment, the fourth hole 132D may be substantially parallel to the first screw hole 132A, and thus extend in a substantially medial-lateral direction in situ. The second and third screw holes 132B, 132C being angled or oblique. For example, the second and third screw holes 132B, 132C may be angled between about ±15 degrees to about ±45 degrees, preferably about ±25 degrees, from the medial-lateral plane (or relative to the first and fourth screw holes 132A, 132D), although other angles may be used.

In addition, and/or alternatively, one or more of the screw holes may be in the form of an elongated slot. In one embodiment, one or more of the screw holes formed in the distal end portion 130 of the retrograde femoral nail 100 may be in the form of an elongated slot. For example, in one embodiment, the second and third screw holes 132B, 132C (e.g., the second and third screw holes 132B, 132C from the distal end of the retrograde femoral nail 100) may be in the form of an elongated slot. Thus arranged, by forming the second and third screw holes 132B, 132C as an elongated slot, the second and third screw slots 132B, 132C may be used to compress a fracture. That is, as will be appreciated by one of ordinary skill in the art, fracture compression may be accomplished by using slotted holes in combination with a reduction instrument. In use, the reduction instrument may be any now known or hereafter developed reduction instrument.

In one embodiment, the reduction instrument includes a reducer, a buttress, and a locking device that engages a proximal bone fragment and a distal bone fragment. In use, the retrograde femoral nail 100 is secured to the distal bone fragment by the locking device. The buttress engages the retrograde femoral nail 100 through an opening (e.g., second or third screw slot 132B, 132C). The reducer includes a compressing screw that applies a force on the buttress, reducing the fracture. Additional information on the reduction instruction can be found in U.S. U.S. Pat. No. 8,628,531, filed Jun. 25, 2008, entitled Assemblies for the Reduction of a Fracture, the entire disclosure of which is hereby incorporated by reference in its entirety.

By utilizing the retrograde femoral nail with one or more elongated slots with a reduction instrument, compression of fibula fractures in the patient's bone may be accomplished.

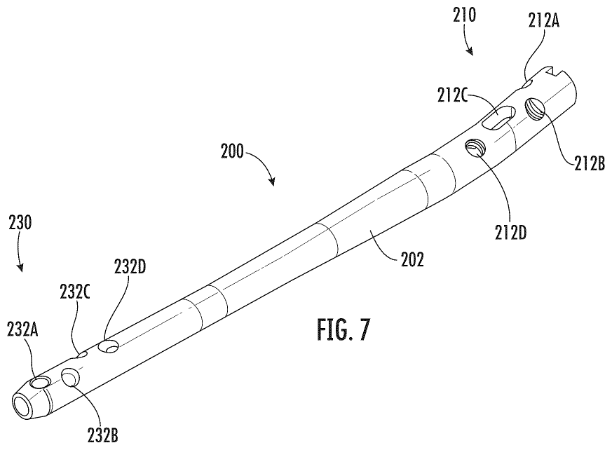

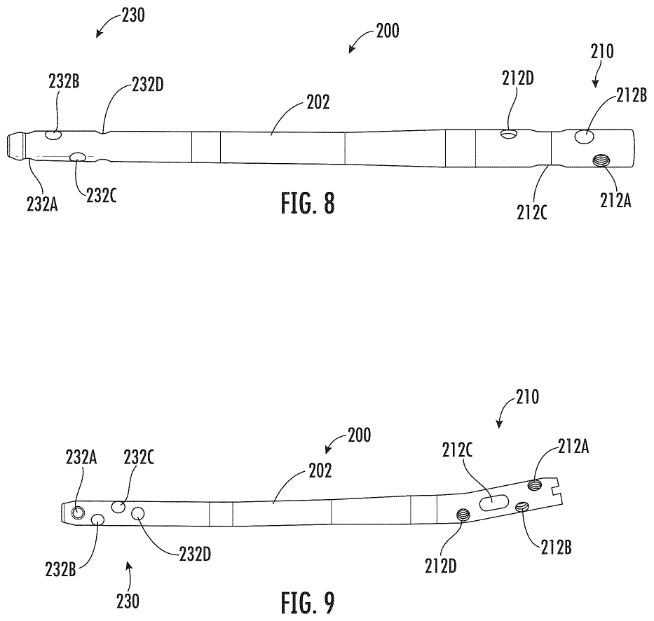

Referring to FIGS. 7-9, an example of an embodiment of a tibial IM nail 200 in accordance with one or more features of the present disclosure is illustrated. In use, as previously mentioned, a tibial IM nail is arranged and configured to be implanted into the medullary canal of a patient's tibia.

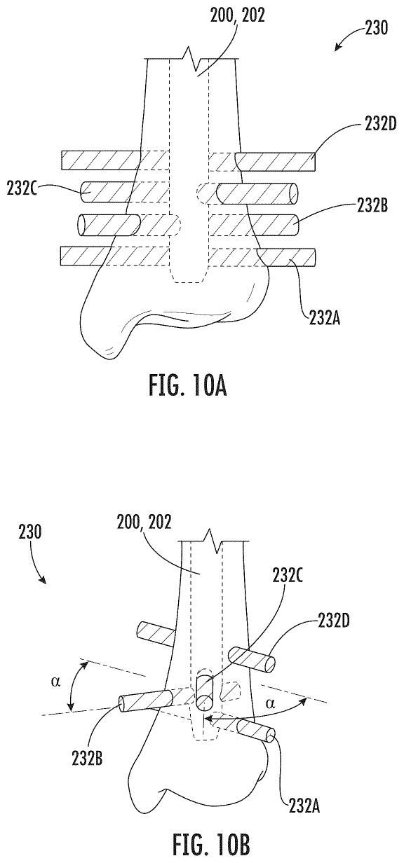

As illustrated in FIGS. 7-9, the tibial IM nail 200 includes a body 202 including a proximal end portion 210 and a distal end portion 230 (e.g., distal end portion 230 now being the leading end portion). As illustrated in FIGS. 7-10B, in one embodiment, the distal end portion 230 includes a plurality of screw openings, holes, slots, etc. 232 arranged and configured to receive a screw. As illustrated, in one embodiment, the distal end portion 230 of the tibial IM nail 200 includes first, second, third, and fourth holes 232A, 232B, 232C, 232D. In use, the second and third screw holes 232B, 232C extend at oblique angles (e.g., second and third screws holes are angled relative to the medial-lateral plane by angle α as best shown in FIG. 10B). As such, as illustrated, the distal end portion 230 of the tibial IM nail 200 includes a first screw hole 232A and a fourth screw hole 232D extending substantially in the medial-lateral direction. Thereafter, the second screw hole 232B and the third screw hole 232C may extend at oblique angles. Thus arranged, by properly orientating the configuration and positioning of the second and third oblique screw holes 232B, 232C, anatomical structures may be avoided.

In one embodiment, the second and third screw holes 232B, 232C may be arranged and configured at an angle α between approximately ±30 to ±60 degrees from medial-lateral in either direction (e.g., rotated approximately ±30 to ±60 degrees relative to the medial-lateral plane).

In addition, in one embodiment, the second and third oblique screw holes 232B, 232C may be tilted (e.g., angled vertically), although the second and third screw holes 232B, 232C may be horizontally positioned (e.g., parallel with respect to the first and fourth screw holes 232A, 232D). That is, one or both of the oblique screw holes 232B, 232C may be angulated relative to a central longitudinal axis of the distal portion of the tibia IM nail 200.

In one embodiment, the first or distal most screw hole 232A may be positioned approximately 6 mm from the distal end of the tibial IM nail 200. The second oblique hole 232B may be positioned approximately 14 mm from the distal end of the tibial IM nail 200. The third oblique screw hole 232C may be positioned approximately 22 mm from the distal end of the tibial IM nail 200. The fourth medial-lateral screw hole 232D may be positioned approximately 30 mm from the distal end of the tibial IM nail 200. However, it should be appreciated that such dimensions are but one example and other dimensions may be used.

Thus arranged, by optimizing the position and angulation of the screw holes, maximum securement may be achieved while avoiding anatomic structures such as, for example, the nerves, vessels, and tendons.

Referring to FIGS. 7-9, 11A, and 11B, in accordance with one or more features of the present disclosure that may be used separately from or in combination with the novel distal end portion discussed above in connection with FIGS. 10A and 10B, the proximal end portion 210 of the tibial IM 200 nail may be arranged and configured with a plurality of screw openings, holes, or slots 212. In one embodiment, the first or most proximal hole 212A is arranged and configured to align a screw approximately parallel with a tibial plateau TP through which the tibial IM nail 200 is inserted. That is, in one embodiment, the first or most proximal screw hole 212A formed in the proximal end portion 210 of the tibia IM nail 200 is arranged and configured to achieve approximate parallel screw alignment with the tibial plateau TP.

As illustrated, in one embodiment, the first or proximal most screw hole 212A formed in the proximal end portion 210 of the tibia IM nail 200 may be tilted or angled downwards away from the proximal end of the tibial IM nail 200. That is, the first or proximal most screw hole 212A may be angled downwards so that a screw inserted therein is positioned substantially parallel to the tibial plateau TP through which the tibia IM nail 200 is inserted. In one embodiment, the first or proximal most screw hole 212A may be angled or tilted downwards by approximately 5 degrees. Alternatively, in alternate embodiments, the first or proximal most screw hole 212A may be angled or tilted downwards by approximately 7 to 10 degrees. Thus arranged, the screw inserted into the first or proximal most screw hole 212A may include a downward slope of between 5 and 10 degrees, plus or minus a degree.

In this manner, the first or proximal most screw hole 212A is arranged and configured to align a screw or other fastener approximately parallel with the tibial plateau TP or, at a minimum, to avoid penetration of the tibial plateau TP.

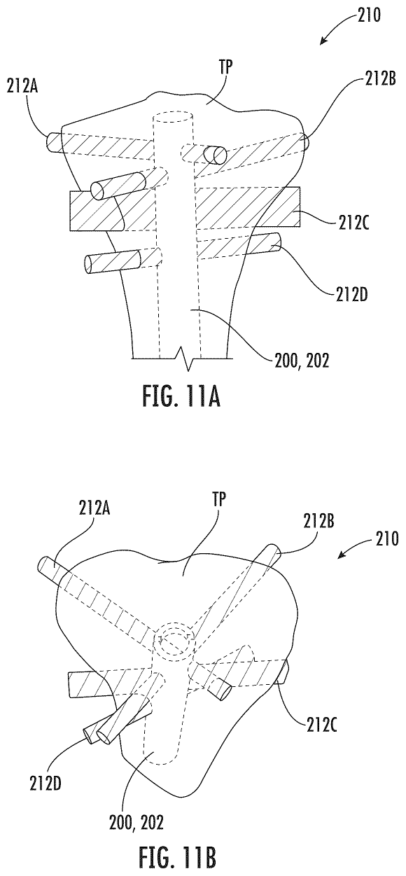

In addition, in one embodiment, the proximal end portion 210 of the tibial IM nail 200 may also include additional screw openings, holes or slots. For example, as illustrated, in one embodiment, the proximal end portion 210 of the tibial IM nail 200 may include a second, third and fourth screw opening, hole, or slot 212B, 212C, 212D. In one embodiment, as best illustrated in FIG. 11A, the second screw hole 212B from the proximal end of the tibia IM nail 200 may also be tilted or angled. In one embodiment, the second screw hole 212B may include a downward tilt or angle. Alternatively, the second screw hole 212B may include an upward tilt or angle. Thus arranged, angulation of the second screw hole 212B facilitates increased bone fixation in the cortical bone (e.g., increased screw lengths may be utilized to achieve increased bone fixation while avoiding penetration of the tibial plateau TP).

In addition, and/or alternatively, in accordance with one or more features of the present disclosure, as illustrated, the third screw hole 212C from the proximal end of the tibial IM nail 200 may be in the form of an elongated slot. Thus arranged, in use, compression at the proximal end portion 210 of the tibial IM nail 200 may be achieved. In addition, and/or alternatively, the second screw hole 212B from the proximal end of the tibial IM nail 200 may be in the form of an elongated slot so that the second screw hole 212B may be used to achieve compression.

Thus arranged, in accordance with one or more features of the present disclosure, by downwardly tilting or angling the first or proximal most first screw hole 212A, the potential for the first or proximal most screw to penetrate the tibial plateau TP is reduced. It is also beneficial to enable the first or proximal most first screw to avoid penetrating the tibial plateau TP without requiring supplemental components be added to the proximal end portion 210 of the tibial IM nail 200 to achieve the angulation required. In addition, the orientation enables improved cortical bone purchase without changing the fundamental structure of and instrumentation requirements for the nail. It is also advantageous to maintain the ability to perform proximal fragment compression using conventional techniques (e.g., incorporation of one or more slots, maintains the ability to achieve compression at the proximal end portion 210 of the tibial IM nail 200. Additionally, the tibial IM nail 200 avoids requiring significant changes to nail instrumentation or insertion methods).

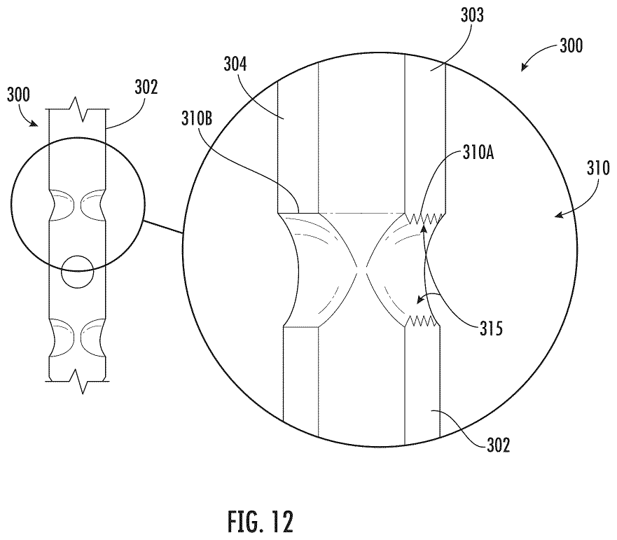

Referring to FIG. 12, in connection with another feature of the present disclosure, that may be used separately or in combination with the other features described herein, a detailed cross-sectional screw hole of an IM nail such as, for example, a retrograde femoral nail 100, a tibial IM nail 200, or any other now known or hereafter developed IM nail is illustrated. In use, the IM nail 300 includes a body 302 (e.g., a cannulated body) including a screw hole 310 arranged and configured to facilitate easier removal of a distal portion of a broken locking screw.

As will be appreciated by one of ordinary skill in the art, inserting a screw through a screw hole formed in a cannulated IM nail requires the screw to pass through a first or proximal screw hole portion formed in a first or proximal wall of the IM nail and through a second or distal screw hole portion formed in a second or distal wall of the IM nail (e.g., proximal and distal referring to a trajectory of the screw path). In use, a locking screw inserted into a threaded screw hole formed in an IM nail may break when the IM nail is subject to loading or during screw insertion through the IM nail and into the patient's bone. However, if the screw is arranged and configured to engage threads formed in the first or proximal screw hole portion formed in the first or proximal wall and the second or distal screw hole portion formed in the second or distal wall, as is the case with known IM nails, removal of the distal portion of the broken locking screw is rendered difficult. That is, in use, a locking screw is arranged and configured to threadably engage both the first and second walls of the IM nail, thus the screw threadably engages the first or proximal screw hole portion and the second or distal screw hole portion. If the screw breaks distally of the first or proximal threaded screw hole portion, as is the usual case, removal of the distal portion of the broken screw is difficult since the distal portion is still threadably coupled to the second or distal threaded screw hole portion and detached from the head portion of the screw. As such, rotating the head portion of the screw does not remove the distal portion of the broken screw.

Presently, one method for removing the broken distal portion of the broken screw from the patient involves tissue removal and grasping and turning of the far distal portion of the screw (e.g., a second incision may be formed in the opposite side of the patient's leg to grasp and rotate the distal portion of the broken screw to threadably decouple it from the second or distal threaded screw hole portion formed in the second or distal wall of the IM nail). Alternatively, the far distal portion of the broken screw may be drilled (e.g., the portion of the screw where the distal portion of the screw contacts the IM nail). In either scenario, each of these solutions is problematic due to the potential for unwanted tissue damage and introduction of unwanted debris into the surgical site.

Thus, it would be beneficial to provide a more practical solution. For example, it would be advantageous to be able to merely drive out the distal portion of the broken screw with a punch or similar tool through the first or proximal hole portion formed in the first or proximal wall of the IM nail left by removing the proximal portion of the broken screw, which may be rotated out. However, this is not possible where the distal portion of the screw remains threadably coupled to the threads formed in the second or distal screw hole portion formed in the second or distal wall of the IM nail (e.g., in instances where the screw breaks distally of the first or proximal screw hole portion formed in the IM nail, the distal portion of the broken screw remains threadably coupled to the second or distal screw hole portion formed in the IM nail). In other words, it is not practical to require a surgeon to strike a proximal end portion of the distal portion of the broken screw with a punch or similar tool with adequate force to shear through the threads coupling the distal portion of the broken screw and the IM nail.

Referring to FIG. 12, in one embodiment, the IM nail includes a first or proximal wall 303 and a second or distal wall 304, the screw hole 310 formed in the IM nail 300 passes through both the first or proximal wall 303 and the second or distal wall 304. However, in accordance with one or more features of the present disclosure, threads 315 for engaging the threads of a locking screw are only provided in the first or proximal wall 303 (e.g., first or proximal screw hole portion 310A) of the IM nail 300 along the trajectory of the locking screw (e.g., the second or distal screw hole portion 310B formed in the second or distal wall 304 is devoid of any threads). Thus arranged, if the screw breaks distally beyond the threads 315 formed in the first or proximal screw hole portion 310A formed in the first or proximal wall 303 of the IM nail 300, the distal portion of the broken screw can be driven out of the patient along the trajectory or path of the screw, and it will only be necessary to overcome the purchase of the distal portion of the broken screw with the tissue in which it is implanted, not the threaded purchase between the distal portion of the broken screw with the second or distal screw hole portion 310B formed in the second or distal wall 304 of the IM nail 300 through which the screw is inserted.

As illustrated, in one embodiment, the first or proximal wall 303 of the IM nail 300 through which the locking screw is initially inserted is threaded (e.g., includes threads 315) while the second or distal wall 304 of the IM nail 300 is non-threaded. In addition, the second or distal screw hole portion 310B formed in the second or distal wall 304 of the IM nail 300 preferably includes a diameter D2 that is greater than a diameter D1 of the threaded portion of the locking screw so that the threaded portion of the screw can pass through the second or distal screw hole portion 310B formed in the second or distal wall 304 of the IM nail 300 (e.g., in FIG. 12, the locking screw is represented by dashed lines for clarity of visualization of the IM nail). Alternatively, in one embodiment, diameter D2 may be equal to diameter D1. In one embodiment, diameter D1 may be approximately 12 mm to 13 mm, diameter D2 may have a lower limit of 12 mm, although these dimensions are exemplary and other dimensions may be used.

Thus arranged, if the locking screw breaks anywhere distal of the first or proximal threaded screw hole portion 310A formed in the first or proximal wall portion 303 of the IM nail 300, the proximal portion of the screw may be backed out using the head of the screw (e.g., the proximal portion of the broken screw can be rotated out of the IM nail 300). The distal portion of the broken screw may be driven distally through the tissue with a punch or other instrument that fits through the second or distal non-threaded hole portion 310B formed in the second or distal wall 304 of the IM nail 300 left by the extracted proximal portion of the broken screw. By this method, the threads of the screw are only engaging tissue (not the IM nail 300 at any location) and the purchase in the tissue may be easily overcome.

In addition, an extractor that includes a hole saw type cutter may be advanced from the distal end of the broken screw to core a tissue plug around the cross-section of the broken screw to more easily remove the screw. With the proposed method, the hole saw type cutter would not have to cut through any implant material, only tissue.

In an alternate embodiment, the distal portion of the screw may also be non-threaded. For example, the distal portion (e.g., shaft portion) of the screw may be in the form of a pin. Thus arranged, easier extraction of the distal portion of the broken locking screw be achievable.

In either event, by arranging the second or distal wall 304 of the IM nail 300 with a non-threaded screw hole portion 310B, easier removal of the distal portion of a broken locking screw is achievable resulting in reduced tissue damage and eliminating, or at least reducing, inadvertent introduction of debris into a surgical site.

Also disclosed herein are various IM nails including one or more features arranged and configured to be implanted within the medullary canal of a patient's tibia. As will be described in greater detail herein, in various examples, the tibial IM nails are arranged and configured to be anatomically specific (e.g., the tibial IM nails include one or more features that are arranged and configured to be positioned in either the patient's left tibia or the patient's right tibia, but not both). This is in contrast to current tibial IM nails on the marketplace, which are arranged and configured to be implanted into both the right and left tibias. Thus arranged, by designing side-specific tibial IM nails, the distal end of the tibial IM nails may be arranged and configured to target one or more specific bony landmarks such as, for example, the patient's posterior malleolus, the syndesmotic joint, etc. while the proximal end of the tibial IM nails may be arranged and configured to target the posterior medial and posterior lateral corners or portions of the patient's tibia.

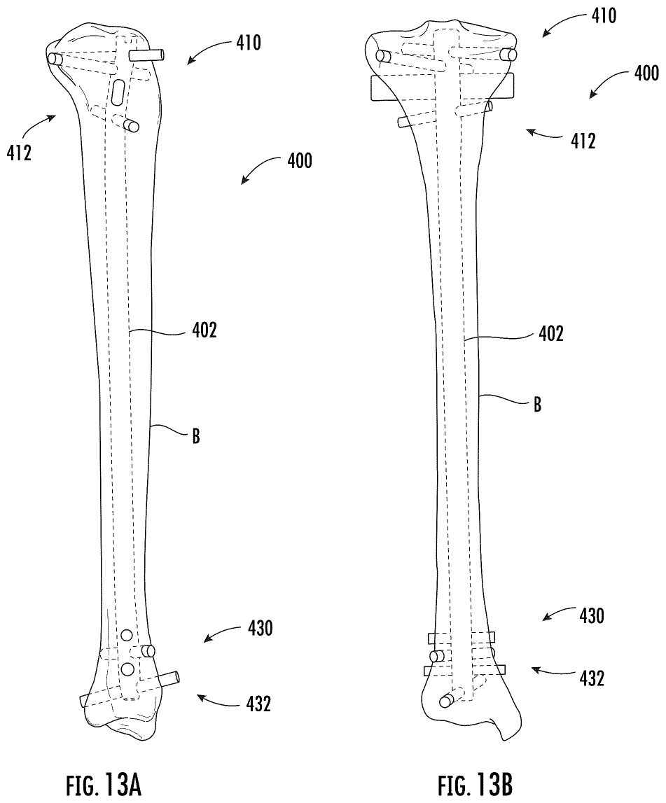

Referring to FIGS. 13A and 13B, an example of a side-specific tibial IM nail 400 in accordance with one or more features of the present disclosure is illustrated. As illustrated, a left-sided tibial IM nail 400 is illustrated implanted within an intramedullary canal of a patient's left tibia B. A right-sided tibial IM nail would be a mirror-image of the left-sided tibial IM nail shown. In use, as previously mentioned, a tibial IM nail 400 is arranged and configured to be implanted into the medullary canal of a patient's tibia. As illustrated, the side-specific tibial IM nail 400 includes a body 402 such as, for example, a cannulated body. The body 402 includes a proximal end portion or region 410 and a distal end portion or region 430 (terms portion or region used interchangeably herein without the intent to limit or distinguish).

As generally illustrated, the distal end portion 430 may include a bow or a bend. In some examples, the bow or bend may be approximately 2 degrees in the anterior-posterior direction, coming from posterior to anterior, starting approximately 60 mm from the distal tip of the tibial IM nail 400, although this is but one configuration and other configurations are envisioned.

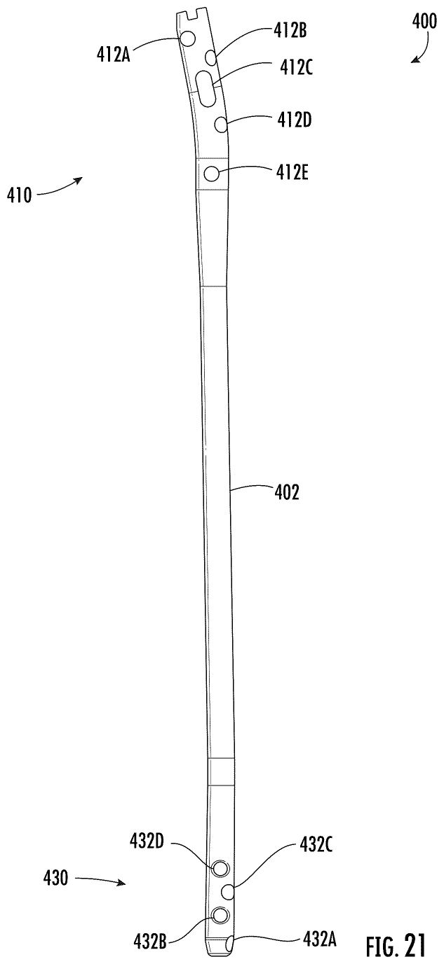

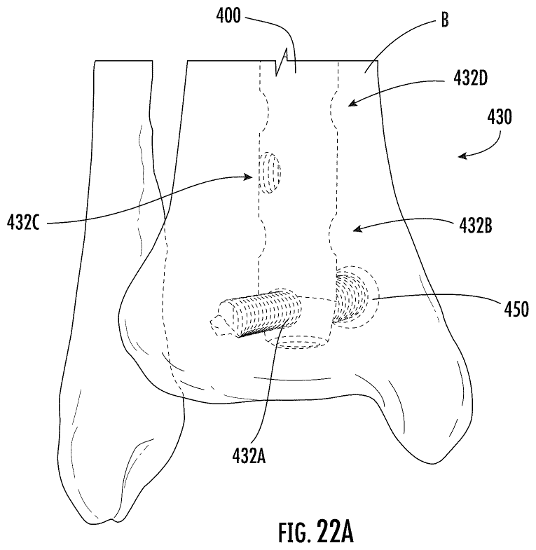

With additional reference to FIG. 21, in some examples, the proximal end portion 410 may include a bow or a bend. In some examples, the bow or bend may extend proximally via an angle of approximately 10 degrees in the anterior-posterior direction, coming anterior from posterior, and located approximately 27 mm from the proximal end of the tibial IM nail 400, although this is but one configuration and other configurations are envisioned.

In accordance with one or more features of the present disclosure, by providing side-specific IM nails, the proximal and distal end portions 410, 430 may also include a bow or a bend in the medial-lateral plane (e.g., the proximal and distal end portions 410, 430 may include a bow or a bend in or out of the medial-lateral plane in addition to, or alternatively from, the bow or bend in the anterior-posterior plane). By providing a bow or bend in the medial-lateral plane, the IM nail is better able to address inherent rotation in the tibia bone of the distal part relative to the proximal part.

In some examples, the proximal end portion 410 may be arranged and configured to couple or receive a nail cap. In some examples, the nail caps may be provided as a set in 5 mm increments starting with 0 mm to 20 mm, although this is but one configuration and other configurations are envisioned.

With additional reference to FIG. 14, the distal end portion 430 includes a plurality of screw openings, holes, etc. 432 (terms used interchangeably herein without the intent to limit) arranged and configured to receive a fastener, screw, etc. (terms used interchangeably herein without the intent to limit) in situ. In some examples, the plurality of screw holes 432 can be threaded. Alternatively, the screw holes can be non-threaded, or some combination of threaded holes and non-threaded holes, or have any other configuration now known or hereafter developed. As illustrated, in some examples, the distal end portion 430 includes first, second, third, and fourth screw holes 432A-432D, although more or less screw holes may be incorporated.

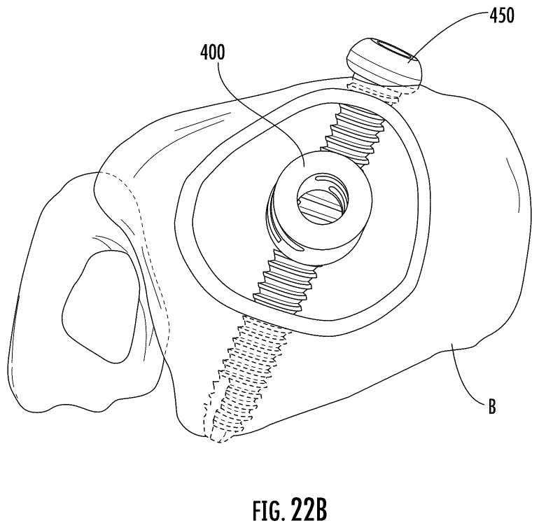

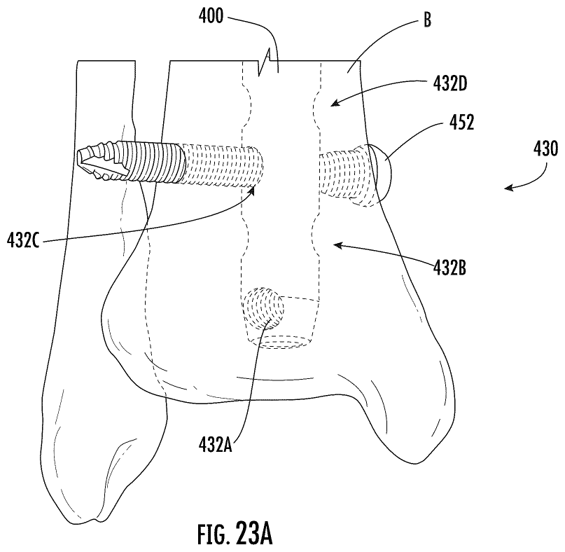

In accordance with one or more features of the present disclosure, the side-specific tibial IM nail 400 includes a distal most, first screw hole 432A that is angled or inclined in the superior-inferior direction relative to a central longitudinal axis of the distal end portion 430. Thus arranged, in use, a screw may be inserted into the distal most, first screw hole 432A and into the patient's posterior malleolus or other bony anatomy. For example, the distal most, first screw hole 432A may have an angle α. In some examples, angle α may be between 8 degrees and 15 degrees, preferably approximately 10 degrees relative to the central longitudinal axis of the distal end portion 430. As such, with additional reference to FIGS. 22A and 22B, a screw 450 may be inserted through the first screw hole 432A formed in the distal end portion 430 of the IM nail 400 and directly into the patient's posterior malleolus. Once again, this is in contrast to current tibial IM nails, which cannot enable targeting of patient specific bony anatomy such as, for example, the patient's posterior malleolus (e.g., the distal most, first screw hole in current tibial IM nails cannot be angled at an approximately 10-degree angle since current tibial IM nails are designed to be implanted into both the patient's left and right tibias). Thus arranged, in use, the IM nail 400 may be used to treat a patient's posterior malleolar fracture. In some examples, in use, a surgeon may select a corresponding side-specific tibial IM nail from a plurality of tibial IM nails including a right tibial IM nail and a left tibial IM nail depending on the location of the fracture (e.g., either the patient's right tibia or left tibia). Thereafter, the surgeon may implant or insert the selected tibial IM nail into the patient's intramedullary canal and target the patient's posterior malleolar fracture via inserting a fastener or screw 450 through the first screw hole 432A formed in the distal end portion 430 of the IM nail 400.

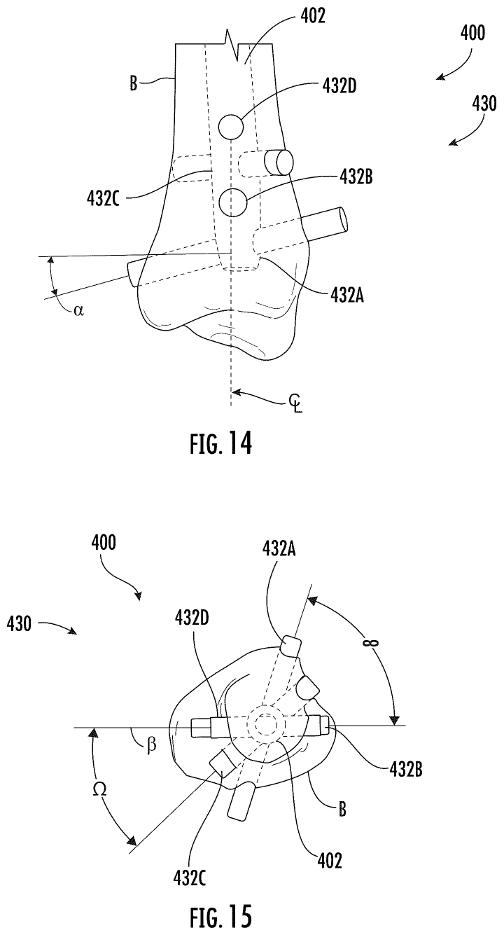

With additional reference to FIG. 15, which illustrates a top-down view of the tibial IM nail 400 implanted within a patient's tibia (e.g., looking proximal to distally), in some examples, the second screw hole 432B and fourth screw hole 432D (e.g., the screw hole positioned second from the distal end and the most proximal screw hole from the distal end of the tibial IM nail 400) may extend substantially parallel to the anterior-posterior plane in situ. Thus arranged, the second screw hole 432B and fourth screw hole 432D may be parallel to each other and to the anterior-posterior plane. In use, by arranging the second and fourth screw holes 432B, 432D parallel to each other and parallel to the anterior-posterior plane, the second and fourth screw holes 432B, 432D are oriented to provide easier targeting such as, for example, freehand targeting of the screw holes 432B, 432D using fluoroscopy, which enables faster surgical times especially for simple shaft fractures. Alternatively, it is envisioned that the second screw hole 432B and fourth screw hole 432D may be provided at an angle β relative to the anterior-posterior plane. In some examples, the angle β may be between 0 degrees and 15 degrees to account for specific amounts of tibial torsion. However, as previously mentioned, in some examples, angle β is preferably zero degrees.

Moreover, in some examples, the distal most, first screw hole 432A may be angled in the medial-lateral direction. For example, the distal most, first screw hole 432A may be angled by an angle ∞. In some examples, angle ∞ may be approximately ±60 degrees to about ±80 degrees, preferably about ±70 degrees and more preferably about ±62.5 degrees, from the anterior-posterior plane (or relative to the second and fourth screw holes 432B, 432D), although other angles may be used. Once again, facilitating targeting of the patient's posterior malleolar and avoiding the patient's syndesmosis joint.

In addition, as illustrated, in some examples, the third screw hole 432C (e.g., the screw hole positioned third from the distal end of the tibial IM nail 400) may be angled in the medial-lateral direction. For example, the third screw hole 432C may be angled by an angle Ω. In some examples, angle Ω may be approximately ±30 degrees to about ±40 degrees, preferably about ±30 degrees, from the anterior-posterior plane (or relative to the second and fourth screw holes 432B, 432D), although other angles may be used. Thus arranged, the third screw hole 432C facilitates screw positioning and/or fixation in a low distal one-third of the fracture that often comes across transverse. In addition, it can facilitate fibular fixation, which could be beneficial in elderly or severely osteoporotic patients.

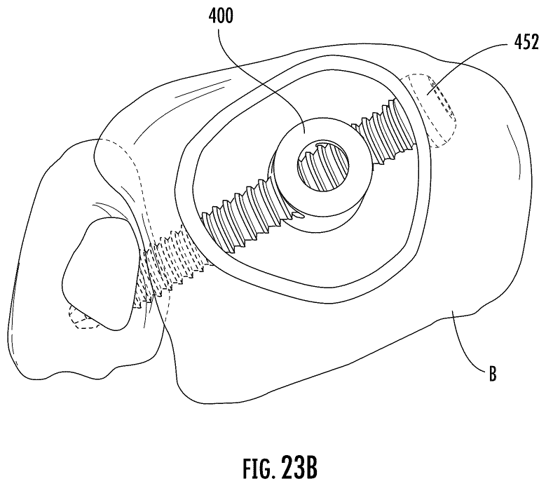

In addition, with additional reference to FIGS. 23A and 23B, a screw 452 may be inserted through the third screw hole 432C formed in the distal end portion 430 of the IM nail 400 into the patient's syndesmotic joint. Once again, this is in contrast to current tibial IM nails, which cannot enable targeting of patient specific bony anatomy such as, for example, the patient's syndesmotic joint (e.g., screw holes in current tibial IM nails cannot be angled at an approximately 30-degree angle since current tibial IM nails are designed to be implanted into both the patient's left and right tibias). Thus arranged, in accordance with one or more features of the present disclosure, the IM nail 400 may be used to treat a patient's syndesmotic joint. In some examples, in use, a surgeon may select a corresponding side-specific tibial IM nail from a plurality of tibial IM nails including a right tibial IM nail and a left tibial IM nail depending on the location of the fracture (e.g., either the patient's right tibia or left tibia). Thereafter, the surgeon may implant or insert the selected tibial IM nail into the patient's intramedullary canal and target the patient's syndesmotic joint via inserting a fastener or screw 452 through the third screw hole 432C formed in the distal end portion 430 of the IM nail 400.

In some examples, in order to account for tibial torsion within the distal end portion 430 of the tibial IM nail 400, an offset may be provided between the third screw hole 432C and the distal most, first screw hole 432A as the distal tibia naturally experiences degrees of torsion that may require surgeons to rotate the tibial IM nail 400 in order to achieve the desired distal fixation, which may result in suboptimal screw placement. By providing side-specific tibial IM nails 400, this can be accommodated by offsetting the third screw hole 432C and the distal most, first screw hole 432A. In some examples, the offset may range from 1-89 degrees to account for the naturally occurring tibial torsion. Alternatively, in an alternate example, in order to account for tibial torsion within the distal end portion 430 of the tibial IM nail 400, an offset may be provided using the second screw hole 432B and the fourth screw hole 432D.

Referring to FIGS. 13A, 13B, 16, and 17, in accordance with one or more features of the present disclosure that may be used separately from or in combination with the distal end portion 430 of the tibial IM nail 400 discussed above in connection with FIGS. 13A-15, the proximal end portion 410 of the tibial IM nail 400 includes a plurality of screw openings, holes, etc. 412 arranged and configured to receive a plurality of screws. As illustrated, in some examples, the proximal end portion 410 of the tibial IM nail 400 includes first, second, third, and fourth holes 412A, 412B, 412C, 412D, although other configurations are envisioned.

As illustrated, in some examples, one or more of the screw holes 412 formed in the proximal end portion 410 of the tibial IM nail 400 may be formed as a slot. For example, as illustrated, the third screw hole 412C from the proximal end portion 410 may be in the form of a slot. By utilizing a slot, dynamization or micro-motion of the tibial IM nail 400 in situ is enabled. In some examples, the third screw hole (e.g., slot) 412C may extend in substantially the medial-lateral direction in situ. In some examples, the slot 412C may have a length of approximately 7 mm.

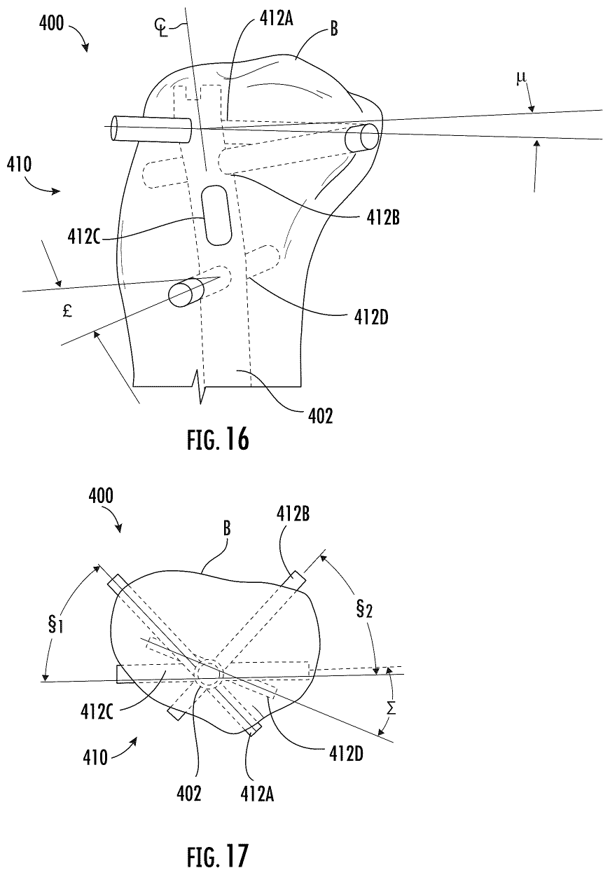

With reference to FIG. 16, the proximal most, first screw hole 412A and the second screw hole 412B are arranged and configured to maximize support of the posterior aspects of the patient's tibia while protecting the knee joint. That is, in accordance with one or more features of the present disclosure, the first and second screw holes 412A, 412B formed in the proximal end portion 410 of the tibial IM nail 400 are arranged and configured to exit the patient's tibia at the same level. For example, as illustrated, the proximal most, first screw hole 412A includes a downward angle or inclination in a superior-inferior direction relative to a central longitudinal axis of the proximal end portion 410. Thus arranged, in use, a screw may be inserted into the proximal most, first screw hole 412A. In situ, the longitudinal axis of the screw positioned within the proximal most, first screw hole 412A crosses the outer surface of the patient's tibia at substantially the same level as a screw positioned within the second screw hole 412B (e.g., plane at which a first screw inserted into the proximal most, first screw hole 412A exits the bone is the same as a second screw inserted into the second screw hole 412B). For example, the proximal most, first screw hole 412A may have an angle or incline of angle μ. In some examples, angle μ may be between 5 degrees and 15 degrees, preferably approximately 10 degrees relative to the central longitudinal axis of the proximal end portion 410 (e.g., a downward ten-degree inclination away from the patient's knee). Meanwhile, the second screw hole 412B may be substantially perpendicular to the central longitudinal axis of the proximal end portion 410. Thus arranged, first and second screws positioned with the first and second screw holes 412A, 412B are arranged and configured to come together, and thus their trajectories can cross or exit the outer surface of the patient's tibia at the same level in situ.

With additional reference to FIG. 17, which illustrates a top-down view of the tibial IM nail 400 implanted within a patient's tibia (e.g., looking proximal to distally), in some examples, the proximal most, first screw hole 412A and the second screw hole 412 are arranged and configured to may be angled in the medial-lateral plane. That is, in some examples, the first and second screw holes 412A, 412B may be angled relative to the medial-lateral plane by an angle § 1, § 2, respectively, and thus relative to the third screw hole (e.g., slot) 412C, which may be arranged and configured to extend in the medial-lateral direction. In some examples, angles § 1, § 2, may be the same. Alternatively, angles § 1, § 2, may be different. In some examples, angle § 1 may be approximately ±40 degrees to about ±50 degrees, preferably about ±44 degrees. Angle § 2 may be approximately ±45 degrees to about ±50 degrees, preferably about ±47.5 degrees. Thus arranged, first and second screws inserted into the first and second screw holes 412A, 412B are better able to target the posterior-medial and posterior-lateral corners or portions of the patient's tibia. In addition, the first and second screws inserted into the first and second screw holes 412A, 412B are better able to avoid the proximal tibial tubercle.

In addition, in some examples, as illustrated in FIG. 16, the fourth screw hole 412D may be angled or inclined in the superior-inferior direction relative to the proximal end portion 410. For example, the fourth screw hole 412D may have an angle £. In some examples, angle £ may be approximately 0 degrees to about ±10 degrees, preferably about ±5 degrees relative to the proximal end portion 410. As such, in some examples, the fourth screw hole 412D may be inclined or declined. Thus arranged, additional opportunities to increase fixation in the event of a more proximal transverse fracture can be achieved.

With additional reference to FIG. 17, in some examples, the fourth screw hole 412D is arranged and configured to may be angled in the medial-lateral plane. That is, in some examples, the fourth screw hole 412D may be angled relative to the medial-lateral plane, and thus relative to the third screw hole (e.g., slot) 412C, which may be arranged and configured to extend in the medial-lateral direction, by an angle Σ. In some examples, angle Σ may be approximately ±25 degrees to about ±35 degrees, preferably about ±25 degrees. Thus arranged, a screw inserted into the fourth screw hole 412D is angled to reduce screwhead prominence on the tibial tubercle.

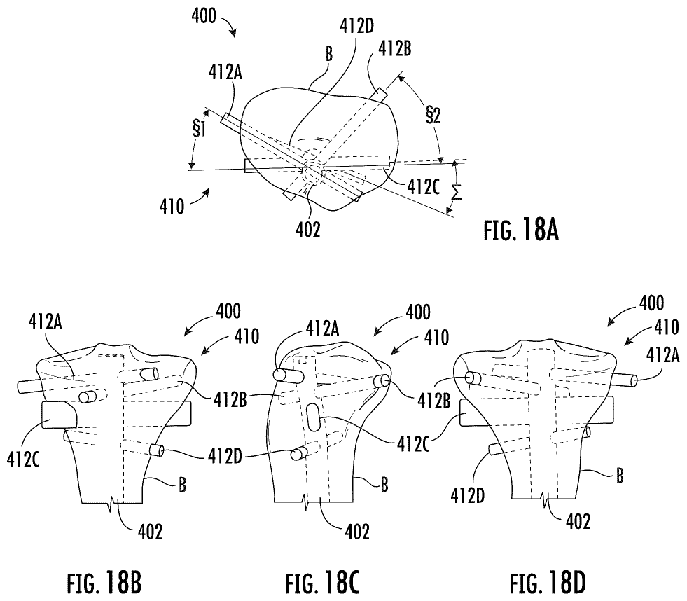

Referring to FIGS. 18A-18D, which illustrates a top-down (e.g., looking proximal to distally), anterior, lateral, and posterior views of an alternate example of a tibial IM nail 400 implanted within a patient's tibia, in some examples, angle § 1 of the proximal most, first screw hole 412A may be ±30 degrees relative to the medial-lateral plane, and thus relative to the third screw hole (e.g., slot) 412C. Thus arranged, the first screw inserted into the first screw hole 412A is arranged and configured to reduce the potential for tissue irritation at the level of the first screw due to the proximal most screw being positioned off the tibial tubercle. In this example, angle § 2 of the second screw hole 412B may be approximately ±47.5 degrees and angle Σ of the fourth screw hole 412D relative to the medial-lateral plane, and thus relative to the third screw hole (e.g., slot) 412C, may be approximately ±25 degrees.

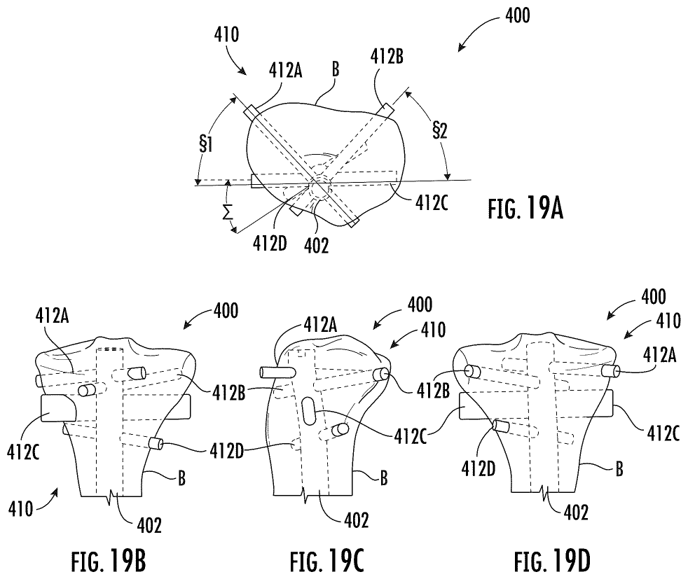

Referring to FIGS. 19A-19D, which illustrates a top-down (e.g., looking proximal to distally), anterior, lateral, and posterior views of an alternate example of a tibial IM nail 400 implanted within a patient's tibia, in some examples, angle E of the fourth screw hole 412D may be angled ±35 degrees to ±40 degrees, preferably, ±40 degrees, relative to the medial-lateral plane, and thus relative to the third screw hole (e.g., slot) 412C. Thus arranged, the fourth screw inserted into the fourth screw hole 412D is arranged and configured to reduce the potential for tissue irritation at the level of the fourth screw due to the fourth screw being rotated off the tibial tubercle, and to increase separation between the third screw positioned within the third screw hole 412C and the fourth screw positioned within the fourth screw hole 412D thereby providing improved fixation when using both a screw positioned within the third screw hole 412C and a screw positioned within the fourth screw hole 412D. That is, in this example, with angle Σ of the fourth screw hole 412D being angled between ±35 degrees to ±40 degrees, preferably, ±40 degrees, relative to the medial-lateral plane, the fourth screw is directed to the postero-lateral aspect of the patient's tibia and changes how the screw head sits as well as its possible interference with other anatomic structures nearby. In contrast, in connection with alternate examples, when angle Σ is angled at ±25 degrees or ±35 degrees, the fourth screw is directed to the postero-medial aspect of the patient's tibia and tends to favor a smaller angle to avoid screw head prominence and maintain safe clearances with surrounding tissue structures.

In this example, angle § 1, § 2 of the first and second screw holes 412A, 412B may both be approximately ±47.5 degrees.

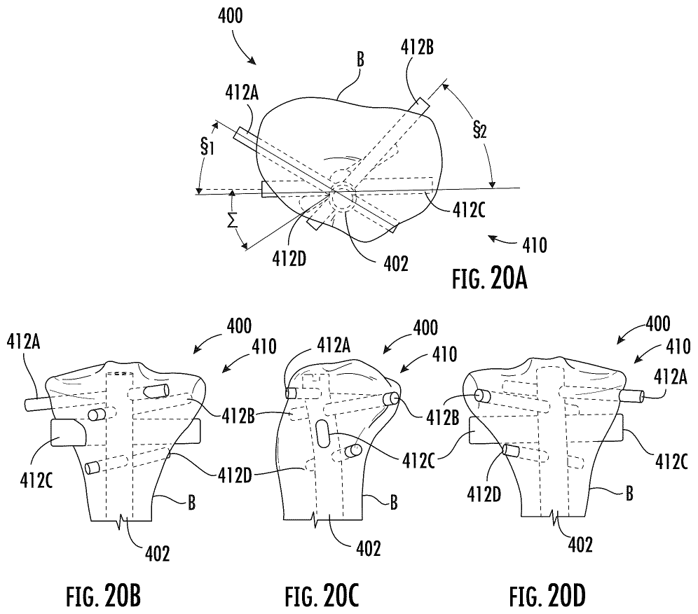

Referring to FIGS. 20A-20D, which illustrates a top-down (e.g., looking proximal to distally), anterior, lateral, and posterior views of an alternate example of a tibial IM nail 400 implanted within a patient's tibia, in some examples, angle § 1 of the proximal most, first screw hole 412A may be ±30 degrees relative to the medial-lateral plane, and thus relative to the third screw hole (e.g., slot) 412C and angle Σ of the fourth screw hole 412D may be angled ±35 degrees relative to the medial-lateral plane, and thus relative to the third screw hole (e.g., slot) 412C. In this example, angle § 2 of the second screw hole 412b may be ±47.5 degrees relative to the medial-lateral plane, and thus relative to the third screw hole (e.g., slot) 412C. Thus arranged, the first screw inserted into the first screw hole 412A is arranged and configured to reduce the potential for tissue irritation at the level of the first screw due to the proximal most screw being positioned off the tibial tubercle and the fourth screw inserted into the fourth screw hole 412E is arranged and configured to reduce the potential for tissue irritation at the level of the fourth screw due to the fourth screw being rotated off the tibial tubercle, and to increase separation between the third screw positioned within the third screw hole 412C and the fourth screw positioned within the fourth screw hole 412D thereby providing improved fixation when using both a screw positioned within the third screw hole 412C and a screw positioned within the fourth screw hole 412D.

Referring to FIG. 21, in accordance with one or more features of the present disclosure, the proximal end portion 410 of the tibial IM nail 400 may include a fifth screw hole 412E. As illustrated, in use, the fifth screw hole 412E is arranged and configured as the most distal of the screw holes formed in the proximal end portion 410. As will be appreciated, the fifth screw hole 412E may be used in combination with any of the tibial IM nails disclosed herein or any tibial IM nail developed hereto or hereafter.

In some examples, as illustrated, the fifth screw hole 412E may be arranged and configured to extend in the medial-lateral plane. Thus arranged, the fifth screw hole 412E is arranged and configured to extend parallel to the medial-lateral plane, and thus parallel to the third screw hole (e.g., slot) 412C, which may be arranged and configured to extend in the medial-lateral direction. In addition, in accordance with one or more features of the present disclosure, the fifth screw hole 412E is separated from the third screw hole 412C (e.g., the two screw holes extending in the medial-lateral plane) via an intermediate screw hole (e.g., the fourth screw hole 412D in the illustrated example). Thus arranged, the fifth screw hole 412E is separated from the third screw hole or compression slot 412C via the fourth screw hole 412D. This is in contrast to current tibial IM nails that include parallel medial-lateral screw holes that are adjacent to each other. By separating the two medial-lateral screw holes from each other, the distance between the two medial-lateral screw holes is increased. In addition, an out-of-plane screw may be positioned between the third and fifth screw holes 412C, 412E. In some examples, the distance between the third and fifth screw holes 412C, 412E may be between 15 mm and 25 mm.

Generally speaking, a large percent of tibia fractures treated with IM nailing are simple shaft fractures and do not require additional out of plane fixation options as provided by the proximal and distal screw trajectories. By incorporating the fifth screw hole 412E and by orientating the fifth screw hole 412E parallel to the third screw hole 412C and to the medial-lateral plane, the tibial IM nail 400 enables surgeons to quickly and more efficiently obtain simple proximal fixation quickly and in the same radiographic orientation as the proximal compression slot, which is medial-lateral oriented as well and commonly used to treat said shaft fractures, thus enabling the surgeon to reduce operative time as well as radiation exposure as less fluoroscopic imaging is needed since the fifth screw hole 412E and the third screw hole (e.g., compression slot) 412C are orientated in the same plane. In addition, by separating the third and fifth screw holes 412C, 412E, enables the tibial IM nail 400 to provide improved fixation by increasing the distance between the two medial-lateral screw holes, as having two screw holes in the same plane adjacent to each other does not provide optimal stabilization and rotational control of fractures.

As such, in accordance with various examples, the IM tibial nail 400 may include first, second, third, fourth, and fifth screw holes 412A-412E in the proximal end portion 410 with two screw holes extending medially and two screw holes extending distally. In various examples, the two proximal extending medial-lateral screw holes and the two distal extending medial-lateral screw holes may be angularly orientated relative to each other (e.g., an angular range between 1 to 89 degrees).

In accordance with one or more features of the present disclosure, a plurality of tibial IM nails may be provided in a kit. For example, a kit may include mutual sets of side-specific anatomic tibial IM nails wherein one tibial IM nail is arranged and configured for implantation into the patient's left tibia and one of the tibial IM nails is arranged and configured for implantation into the patient's right tibia. In use, the tibial IM nails may be mirror-images of each other. In accordance with one or more features of the present disclosure, the tibial IM nails may be color-coded to distinguish the tibial IM nails configured for implantation in the patient's right tibial from the tibial IM nails configured for implantation in the patient's left tibial.

The foregoing description has broad application. Accordingly, the discussion of any embodiment or example is meant only to be explanatory and is not intended to suggest that the scope of the disclosure, including the claims, is limited to these embodiments or examples. In other words, while illustrative embodiments or examples of the disclosure have been described in detail herein, it is to be understood that the inventive concepts may be otherwise variously embodied and employed, and that the appended claims are intended to be construed to include such variations, except as limited by the prior art.

The term “a” or “an” entity, as used herein, refers to one or more of that entity. As such, the terms “a” (or “an”), “one or more” and “at least one” can be used interchangeably herein. The use of “including,” “comprising,” or “having” and variations thereof herein is meant to encompass the items listed thereafter and equivalents thereof as well as additional items. Accordingly, the terms “including,” “comprising,” or “having” and variations thereof are open-ended expressions and can be used interchangeably herein. The phrases “at least one”, “one or more”, and “and/or”, as used herein, are open-ended expressions that are both conjunctive and disjunctive in operation.

All directional references (e.g., proximal, distal, upper, underside, lower, upward, downward, left, right, lateral, longitudinal, front, back, top, bottom, above, below, vertical, horizontal, radial, axial, clockwise, and counterclockwise) are only used for identification purposes to aid the reader's understanding of the present disclosure, and do not create limitations, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority but are used to distinguish one feature from another. The drawings are for purposes of illustration only and the dimensions, positions, order, and relative sizes reflected in the drawings attached hereto may vary.

What is claimed is:

1. An anatomic, side-specific tibial IM nail comprising:
a body including a proximal end portion and a distal end portion, the distal end portion including a plurality of screw holes arranged and configured to receive a fastener, the plurality of screw holes including a distal most, first screw hole;
wherein the distal most, first screw hole is angled in a superior-inferior direction relative to a central longitudinal axis of the distal end portion.

2. The anatomic, side-specific tibial IM nail of claim 1, wherein the distal most, first screw hole is angled by an angle α with angle α being approximately ten-degrees so that a first screw positioned within the distal most, first screw hole can target a patient's posterior malleolus.

3. The anatomic, side-specific tibial IM nail of claim 1, wherein the plurality of screw holes formed in the distal end portion further include second, third, and fourth screw holes positioned from a distal end of the body.

4. The anatomic, side-specific tibial IM nail of claim 3, wherein the second screw hole and the fourth screw hole extend substantially parallel to each other.

5. The anatomic, side-specific tibial IM nail of claim 3, wherein the second screw hole and the fourth screw hole extend substantially parallel to an anterior-posterior plane in situ.

6. The anatomic, side-specific tibial IM nail of claim 3, wherein the distal most, first screw hole is angled by an angle ∞ relative to second and fourth screw holes, wherein the angle ∞ is about ±62.5 degrees.

7. The anatomic, side-specific tibial IM nail of claim 3, wherein the third screw hole is angled by an angle Ω relative to second and fourth screw holes, wherein the angle Ω is about ±30 degrees.

8. The anatomic, side-specific tibial IM nail of claim 1, wherein the proximal end portion includes a plurality of screw holes arranged and configured to receive a fastener, the plurality of screw holes including a proximal most, first screw hole;
wherein the proximal most, first screw hole includes a downward angle of angle μ in a superior-inferior direction.

9. The anatomic, side-specific tibial IM nail of claim 8, wherein the angle μ is approximately 10 degrees.

10. The anatomic, side-specific tibial IM nail of claim 8, wherein the proximal end portion further includes a second screw hole positioned from a proximal end of the body, the first and second screw holes being arranged and configured to exit from an outer surface of a patient's tibia at substantially a same level or plane.

11. The anatomic, side-specific tibial IM nail of claim 10, wherein the second screw hole is substantially perpendicular to a central longitudinal axis of the proximal end portion.

12. The anatomic, side-specific tibial IM nail of claim 10, wherein the proximal end portion further includes third and fourth screw holes positioned from the proximal end of the body.

13. The anatomic, side-specific tibial IM nail of claim 12, wherein one or more of the plurality of screw holes is arranged and configured as a slotted hole.

14. The anatomic, side-specific tibial IM nail of claim 12, wherein the third screw opening is arranged and configured as a slotted hole.

15. The anatomic, side-specific tibial IM nail of claim 12, wherein the proximal most, first screw hole and the second screw hole are angled relative to the third screw hole by an angle § ₁, § ₂, respectively.

16. The anatomic, side-specific tibial IM nail of claim 15, wherein the angles § ₁, § ₂ are about ±47.5 degrees.

17. The anatomic, side-specific tibial IM nail of claim 15, wherein the angle § ₁ is different from the angle § ₂, wherein the angle § ₁ is about ±44 degrees and angle § ₂ is about ±47.5 degrees.

18. The anatomic, side-specific tibial IM nail of claim 12, wherein the fourth screw hole is angled in a superior-inferior direction by an angle £, the angle £ is about ±5 degrees.

19. The anatomic, side-specific tibial IM nail of claim 18, wherein the fourth screw hole is angled relative to the third screw hole by an angle Σ, the angle Σ being selected from one of about ±25 degrees, about ±35 degrees, and about ±40 degrees.

20. The anatomic, side-specific tibial IM nail of claim 12, wherein the proximal end region includes a fifth screw hole.

21. The anatomic, side-specific tibial IM nail of claim 20, wherein the fifth screw hole is parallel to the third screw hole.

22. The anatomic, side-specific tibial IM nail of claim 20, wherein the third screw hole and the fifth screw hole are separated from each other by the fourth screw hole positioned between the third and fifth screw holes.

23. An anatomic, side-specific tibial IM nail comprising:
a body including a proximal end portion and a distal end portion, the proximal end portion including a plurality of screw holes arranged and configured to receive a fastener, the plurality of screw holes including a proximal most, first screw hole;
wherein the proximal most, first screw hole includes a downward angle of angle μ in a superior-inferior direction.

24. A method for treating a patient's posterior malleolar fracture, the method comprising:
selecting a corresponding side-specific tibial IM nail from a plurality of tibial IM nails including a right tibial IM nail and a left tibial IM nail depending on a location of the fracture;
implanting the selected side-specific tibial IM nail into the patient's intramedullary canal; and
inserting a fastener through a screw hole formed in a distal end portion of the side-specific tibial IM nail, the fastener targeting the patient's posterior malleolar fracture.
